(19) United States
(12) Patent Application Publication
(10) Pub. No.: US 2017/0334962 A1
(43) Pub. Date: Nov. 23, 2017

(54) FcRn-TARGETED ANTIGEN FUSION PROTEINS

(71) Applicant: Texas A&M University System, College Station, TX (US)

(72) Inventors: E. Sally Ward Ober, College Station, TX (US); Dilip K. Challa, College Station, TX (US)

(21) Appl. No.: 15/600,079

(22) Filed: May 19, 2017

Related U.S. Application Data
(60) Provisional application No. 62/338,934, filed on May 19, 2016.

(51) Int. Cl. C07K 14/47; A61K 39/00; C07K 14/52

(52) U.S. Cl. CPC C07K 14/4713; C07K 14/52; A61K 39/0008; A61K 2039/577; C07K 2319/30; A61K 2039/6056

(57) ABSTRACT

The invention disclosed herein generally relates to fusion proteins for use alone or as adjuvants or antigen delivery vehicles for vaccines.

### CROSS REFERENCE TO RELATED APPLICATIONS

This application claims the benefit of U.S. Provisional Application No. 62/338,934, filed on May 19, 2016, entitled FcRn-Targeted Antigen Fusion Proteins. The entire content of the foregoing is hereby incorporated by reference.

### FIELD OF THE INVENTION

The invention disclosed herein generally relates to fusion proteins for use alone or as adjuvants or antigen delivery vehicles for vaccines.

### BACKGROUND

Organ-specific autoimmune diseases such as multiple sclerosis (MS), rheumatoid arthritis and type 1 diabetes mellitus represent a major cause of death in developed countries. It is known that the aberrant activation of autoreactive CD4+ T cells is a driver of autoimmune disorders. Currently approved therapies for autoimmunity that broadly target such cells include the depletion of lymphocyte subsets, the targeting of immune activation/co-stimulatory signals or the inhibition of leukocyte trafficking [1]. However, these approaches can result in adverse side effects such as systemic toxicities and increased risk for infection or cancer [1]. Consequently, a need for the development of treatments, such as tolerance induction, to selectively target autoantigen-specific T cells persists.

The induction of autoantigen-specific T cell tolerance using high doses of soluble immunodominant peptides to delete or anergize autoreactive T cells has been explored [2, 3]. Although such approaches, including the delivery of altered peptide ligands, have shown efficacy in reducing disease in animal models of MS and diabetes, the translation of such therapies into humans has been unsuccessful [2, 4]. Further, there are significant safety concerns due to reports of fatal anaphylaxis in many animal models of MS following the delivery of relatively high doses (necessitated by rapid renal clearance [5]) of autoantigenic peptides during ongoing disease [6, 7]. A longstanding, unsolved challenge is therefore to develop effective tolerizing agents that are safe for the therapy of autoimmunity.

Likewise, infectious disease and cancer are among the leading causes of death in the world. For example, in 2014, around 1.2 million people died of AIDS-related illnesses, worldwide (UNAIDS). Also, 589,430 cancer deaths are estimated in the United States alone in 2015 (American Cancer Society). Hence, there is an urgent need for improving the efficacy of existing vaccines or developing new, potent vaccines for the prevention or treatment of cancer and infectious disease.

Various aspects and embodiments of the invention disclosed herein relate to the development of agents that can be used to for the therapy of autoimmunity, cancer, infectious diseases, and the like.

### SUMMARY OF THE INVENTION

Some embodiments of the invention relate to an immune stimulant that can be used alone or in conjugation with vaccine formulations and that comprises a ligand-antigen fusion protein, where the ligand is capable of binding to a Fc receptor (FcRn). Some embodiments relate to ligand-antigen fusion protein that can induce or potentiate immune activation, including the expansion of antigen-specific T cells, wherein the ligand is capable of binding to a Fc receptor (FcRn).

In some embodiments, the ligand of the fusion protein can be a Fc, an IgG, a single chain Fv, a nanobody, any other protein that binds to FcRn, or the like. In some embodiments, the ligand is engineered. For example, the ligand can be engineered so that it binds to the FcRn in a pH-independent manner.

In some embodiments, the ligand can be or can have an engineered Fc region.

In some embodiments, the antigen of the fusion protein can be or can have an immunodominant epitope. For example, the immunodominant epitope can be a myelin basic protein peptide (MBP1-9), chicken egg white ovalbumin (OVA) peptide, or the like. Other potential antigens can include: 1) any cancer-associated antigen including but not limited to overexpressed antigens [e.g., human epidermal growth factor receptor 2 (Her2)], cancer testis antigens [e.g., NY-ESO-1], oncoviral antigens [human papilloma virus (HPV) E6, E7], oncofetal antigens [e.g., tumor antigen-72 (TAG-72)], lineage restricted antigens [e.g., Gp100/pme117], mutated antigens [cyclin-dependednt kinase-4 (CDK4)], posttranslationally altered antigens [e.g., MUC1], idiotypic antigens [e.g., immunoglobulin] or the peptides derived from these antigens; 2) any antigen associated with infectious agents including but not limited to Human Immunodeficiency Virus (HIV), Hepatitis C Virus (HCV), Mycobacterium Tuberculosis (Mtb) and Plasmodium Falciparum.

In some embodiments, multiple different antigens can be fused to the ligand, multiple immunodominant epitopes can be fused to the ligand and/or multiple immunodominant epitopes and antigens can be fused to the ligand.

Some embodiments of the invention relate to an antigen delivery vehicle for vaccines comprising a ligand-antigen fusion protein, where the ligand is capable of binding to a Fc receptor (FcRn).

Some embodiments of the invention relate to methods of treating a subject with a disease treatable with a vaccine where the method includes administration of a ligand-antigen fusion protein. In some embodiments, the disease can be an infectious disease, cancer, or the like.

In some embodiments, the ligand-antigen fusion can be administered as one or more doses following administration of the vaccine. In some embodiments, the ligand-antigen fusion can be administered as one or more doses prior to administration of the vaccine. In some embodiments, the ligand-antigen fusion can be administered as one for more doses prior to and following administration of the vaccine. Likewise, in some embodiments, the ligand-antigen fusion can be administered simultaneously with the vaccine, and/or can be administered before and/or after the vaccine.

In some embodiments of the invention, administration of the ligand-antigen fusion can result in T cell expansion and/or activation.

Some embodiments of the invention relate to a ligand-antigen fusion containing a Toll-like receptor pattern recognition receptor ligand (TLR)-agonist that induces or potentiates immune activation. Some embodiments of the invention relate to a ligand-antigen fusion containing a cytokine that induces or potentiates immune activation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 depicts results from experiments that demonstrate that IgGs or Fc-MBP fusions containing m-set-1 and m-set-2 mutations are cleared more rapidly in mice compared with their WT counterparts. B10.PL mice (n=4-5 mice/group) were injected with ¹²⁵I-labeled IgGs (A, B) or Fc-MBP fusion (C). (A) Remaining radioactivity levels in blood samples. (B, C) Areas under the curve (AUCs, cpm h), calculated for fitted data following extrapolation to 1% injected dose. Error bars indicate SEM and significant differences (p<0.05; two-tailed Student's t-test) are indicated by *.

FIG. 2 depicts results from experiments that demonstrate that in vivo persistence governs the response of cognate T cells to Fc-MBP fusions. (A) Flow chart describing the experimental design. B10.PL mice were injected with 1 μg Fc-MBP fusion 0, 3 and 5 days before the transfer of CFSE-labeled antigen-specific (Vβ8⁻) T cells. CD4⁺CFSE⁺Vβ8⁺ T cell proliferation was analyzed three days later by flow cytometry. (B) % divided Vβ8⁺CFSE⁺ T cells of total CD4⁺ cells in spleens and lymph nodes (LNs) for the different treatments, normalized to the group injected with Fc(long)-MBP on day 0. Data are combined from at least two independent experiments (n=3-4 mice/group). Error bars indicate SEM and significant differences (p<0.05; two-tailed Student's t-test) are indicated by *.

FIG. 3 depicts results from experiments that demonstrate that prophylactic tolerance induction is determined by antigen persistence. (A) B10.PL mice were pretreated with 1 μg Fc-MBP fusion and immunized seven days later with MBP1-9 to induce EAE. Mean clinical scores are shown. Data are combined from at least two independent experiments (n=13-30 mice/group). (B) IL-2 production by antigen-specific T cell hybridoma (#46 [26]) cells in response to the Fc-MBP fusions in the presence of I-A^{u}-expressing PL8 or PL8:FcRn [10] cells. Data is representative of at least two independent experiments. (C) B10.PL mice were pretreated with either 5 doses of 1 μg of Fc(v.short)-MBP (starting at 7 days prior to immunization, at 36 hour intervals) or with a single bolus dose of 5 μg of Fc(v.short)-MBP and immunized seven days later to induce EAE. Mean clinical scores are shown. Data are combined from at least two independent experiments (n=18-26 mice/group). Error bars indicate SEM and significant differences (p<0.05; linear mixed effects model) are indicated by *.

FIG. 4 depicts results from experiments that demonstrate that prophylactic tolerance induction is accompanied by lower numbers of antigen-specific T cells. (A, B, C, D) Quantitation of antigen-specific T cells in the spleens of mice using fluorescently labeled MBP1-9(4Y):I-A^{u} tetramers ten days following immunization. % (boxed, A, D) and total numbers of CD4⁺tetramer⁺ T cells (B, C) are shown. Percentages (±SEM) of CD4⁺ T cells for mice treated with the Fc-MBP fusions were: Fc(long)-MBP, 10.5±0.9; Fc(short)-MBP, 11.4±0.3; Fc(v.short)-MBP, 12.4±0.8; Fc(long)-MBP(3A6A), 10.3±0.6; 5 μg Fc(v.short)-MBP, 10.6±0.7; 5×1 μg Fc(v.short)-MBP, 11.2±0.4. Dot plots show data for one representative mouse within each group (A) or all the mice (D), and data in (B) and (C) are derived from 4-7 mice/group. Error bars indicate SEM and significant differences (p<0.05; two-tailed Student's t-test) are indicated by *. N.S., no significant difference. In FIGS. 3C and 4C,D, a single dose (5 ug) of Fc(v.short)-MBP does not induce tolerance and induces the expansion of much higher numbers of antigen-specific T cells following immunization compared with repeated, lower doses (5×1 μg) of the same Fc-MBP fusion.

FIG. 5 depicts results from experiments that demonstrate that a threshold persistence level of Fc-MBP fusion is necessary for the treatment of EAE. (A, B) B10.PL mice were immunized with MBP1-9 and treated with 1 μg Fc-MBP fusion or 33 ng MBP1-9(4Y) peptide following the onset of disease symptoms (EAE score of 1-2). Mean clinical scores are shown. Data are combined from at least two independent experiments (n=9-26 mice/group). Error bars indicate SEM and significant differences (p<0.05; linear mixed effects model) are indicated by *.

FIG. 6 depicts results from experiments that demonstrate that tolerance induction during ongoing EAE results in increased numbers of peripheral antigen-specific CD4⁺ T cells with downregulated T-bet and CD4OL levels combined with reduced inflammatory infiltrates in the CNS. B10.PL mice were immunized and treated with Fc(long)-MBP as in FIG. 5. Six days following treatment, mice were sacrificed and tissues isolated for flow cytometry analyses to determine: (A) % (in spleens) and total numbers (in spleens, LNs) of CD4⁺tetramer⁺ T cells; (B, C) % (in spinal cords) and total numbers (in brains, spinal cords) of mononuclear infiltrates that are CD4⁺tetramer⁺ T cells (B) or F4/80⁺CD45^{hi} macrophages (C); (D) MFI levels for T-bet amongst CD4⁻tetramer⁺ T cells in spleens and LNs; (E) % CD4⁺tetramer⁺CD40L^{hi} T cells in spleens; (F) % (in spleens) and total numbers (in spleens, LNs) of CD4⁺Foxp3⁺ T cells; (G) Treg (CD4⁺Foxp3⁺ T cells):Th1 (CD4⁺tetramer⁺T-bet⁺ T cells) ratios in spleens and LNs. For A-F, left panels show data for one representative mouse from each group. For A-C, F, populations of interest are indicated in dot plots by solid circles or boxes. Percentages (±SEM) of CD4⁺ T cells for mice treated with the Fc-MBP fusions were: Fc(long)-MBP, 8.8±0.4 (spleens) and 34.8±1.5 (LNs); Fc(long)-MBP(3A6A), 14.7±0.9 (spleens) and 37.5±2.4 (LNs). Data are combined from at least two independent experiments (n=5-8 mice/group; right panels). Error bars indicate SEM and significant differences (p<0.05; two-tailed Student's t-test) are indicated by *.

FIG. 7 depicts results from experiments that demonstrate that prophylactic treatment with Fc(TT-HN)-OVA(323-339) is accompanied by higher percentage of antigen-specific T cells. C57BL/6J mice were pretreated with 25 μg Fc-OVA(323-339) fusions and immunized seven days later with OVA. Eighteen days post-immunization, mice were sacrificed and spleens isolated for flow cytometry analyses to determine the percentage of CD4+OVA(329-337)-I-A(b) tetramer+ T cells. The dot plots are gated on live, B220-CD44+ cells. The populations of interest are indicated in the dot plots by solid boxes.

FIG. 8 depicts results from experiments that demonstrate that prophylactic treatment with Fc(TT-HN)-OVA(323-339) is accompanied by higher numbers of antigen-specific T cells. Total numbers of splenic CD4+CD44+OVA(329-337)-I-A(b) tetramer+ T cells calculated using the data in FIG. 7 are shown.

FIG. 9 depicts results from experiments that demonstrate that Fc-MBP fusions do not affect the clearance rate of mouse IgG1. B10.PL mice (n=4-5 mice/group) were injected with ¹²⁵I-labeled mouse IgG1. 24 hours later (indicated by arrow in panel A), the mice were injected with DPBS or 1 μg Fc-MBP fusion. (A) Remaining radioactivity levels in blood samples. (B) β-phase half-lives of mouse IgG1, calculated for fitted data. Error bars indicate SEM. N.S., no significant difference (p >0.05; two-tailed Student's t-test).

FIG. 10 depicts results from experiments that demonstrate that prophylactic tolerance induction does not result in increased numbers of CD4⁺Foxp3⁺ T cells. B10.PL mice were treated as in FIG. 3. (A, B) Numbers of CD4⁺Foxp3⁺ T cells in the spleens were determined using flow cytometry ten days following immunization of mice with MBP1-9. Data are derived from 4-7 mice per treatment group. Error bars indicate SEM. N.S., no significant difference (p>0.05; two-tailed Student's t-test).

FIG. 11 depicts results from experiments that demonstrate that Tolerance induction during ongoing EAE results in increased numbers of peripheral antigen-specific CD4⁺ T cells with downregulated T-bet and CD4OL levels combined with reduced inflammatory infiltrates in the CNS. B10.PL mice were immunized and treated with Fc(short)-MBP as in FIG. 6. Six days following treatment, mice were sacrificed and tissues isolated for flow cytometry analyses to determine: (A) % (in spleens) and total numbers (in spleens, LNs) of CD4⁺tetramer⁺ T cells; (B, C) % and total numbers of mononuclear infiltrates in the spinal cords that are CD4⁺tetramer⁺ T cells (B) or F4/80⁺CD45^{hi} macrophages (C); (D) MFI levels for T-bet amongst CD4⁺tetramer⁺ T cells in spleens and LNs; (E) % CD4⁺tetramer⁺CD40L^{hi} T cells in spleens; (F) % (in spleens) and total numbers (in spleens, LNs) of CD4⁺Foxp3⁺ T cells; (G) Treg (CD4⁺Foxp3⁻T cells):Th1 (CD4⁺tetramer⁺T-bet⁺ T cells) ratios in spleens and LNs. For A-F, data for one representative mouse from each treatment group is presented in the left panels. For A-C, F, populations of interest are indicated in dot plots by solid circles or boxes. Percentages (±SEM) of CD4⁺ T cells for mice treated with the Fc-MBP fusions were: Fc(short)-MBP, 9.5±0.8 (spleens) and 28.8±0.8 (LNs); Fc(long)-MBP(3A6A), 11.5±0.6 (spleens) and 19.7±2.7 (LNs). Data are derived from 3-4 mice/group (A-C, E-G) or 7 mice/group (D). Error bars indicate SEM and significant differences (p<0.05; two-tailed Student's t-test) are indicated by *.

### DETAILED DESCRIPTION OF THE INVENTION

Unless otherwise noted, terms are to be understood according to conventional usage by those of ordinary skill in the relevant art.

Chronic exposure to autoantigens during autoimmunity results in reduced disease severity, with mouse studies indicating that this phenomenon results from regulatory T cell (Treg) activation [8]. In addition, low dose, persistent antigen presentation during chronic viral infections can lead to CD4⁺ T cell exhaustion or dysfunction in an antigen-specific manner [9]. Embodiments of the invention relate to the development and use of delivery vehicles to enable persistence of low levels of antigen as an effective approach to induce antigen-specific T cell tolerance. However, the generation of antigen delivery strategies to achieve such immune homeostasis is challenging due to the limited understanding of the complex interplay between antigen longevity and intracellular trafficking behavior, which in turn determines the efficiency of antigen presentation by antigen presenting cells (APCs).

Aspects of the invention relate to Fc engineering studies that indicate that antigenic peptide epitopes expressed as immunoglobulin Fc-epitope fusions can be tuned to have different pharmacokinetics by modulating their binding properties for the neonatal Fc receptor (FcRn) [10]. The majority of naturally occurring antibodies of the IgG class bind to FcRn at acidic pH (pH 6.0) but with an affinity that is negligible at near neutral pH [11]. Consequently, following entry into cells bathed at pH 7.3-7.4 by fluid phase processes, IgG can bind to FcRn in early acidic endosomes and undergo recycling or transcytosis [11-13]. These endosomal sorting pathways regulate the homeostasis and transport of IgG in the body. Further, FcRn is expressed in all professional APCs and is involved in antigen presentation [14]. Embodiments of the invention relate to designing a panel of Fc-epitope fusions comprising the N-terminal epitope of myelin basic protein (MBP1-9) linked to engineered Fc regions to define the requirements for tolerance induction in a low antigen dose setting. Specifically, Fc-MBP fusions can be generated with different subcellular trafficking behavior and in vivo clearance properties. For example, in some embodiments of the invention, engineered proteins can effect both the prophylactic blockade and treatment of disease in an EAE model involving the immunization of B10.PL (H-2^{u}) mice with the immunodominant epitope, MBP1-9 (with N-terminal acetylation). Other embodiments of the invention relate to fusions comprising recombinant Fc fragments fused to chicken egg white ovalbumin (OVA) peptide. Prophylactic treatment with FcRn-targeted Fc-antigen (OVA peptide) fusion protein can potentiate vaccination-induced antigen (OVA)-specific CD4+ T cell response.

In some embodiments of the invention, Fc-engineering can tune antigen dynamics to establish the design requirements for antigen delivery vehicles that result in T cell tolerance and amelioration of ongoing autoimmune disease. Some embodiments related to using doses (1 μg/mouse; ˜50 μg/kg) that are at least ˜450-fold lower than those used previously as either soluble antigen or peptides coupled to microparticles for the treatment of autoimmunity [3, 15-17], reducing the risk of anaphylactic shock. Embodiments of the invention relate to a remarkably stringent threshold of antigen persistence that is effective to induce tolerance prior to disease induction and during ongoing disease. In these two settings, although the threshold for antigen persistence is the same, the pathways of tolerance induction are mechanistically distinct: under prophylactic conditions, antigen-specific T cells are deleted or anergized whereas during ongoing EAE, tolerance involves the downregulation of T-bet and CD40L on antigen-specific T cells, combined with the induction of regulatory Foxp3⁺ T cells. Embodiments of the invention relate to the delivery of low doses of Fc-epitope fusions represents a promising strategy for the treatment of autoimmunity and other pathological, T cell-mediated conditions.

Embodiments of the invention disclosed herein describe the use of xfcrn-antigen fusion proteins as ‘adjuvants’ or ‘antigen delivery vehicles’ for vaccines, where ‘xfcrn’ denotes Fc, IgG, single chain Fv, a nanobody or any other ligand that can bind to the Fc receptor (FcRn) in a pH-independent fashion (generally between pH 4.5 and 8, and particularly at pH 5.5-6 and pH 7-7.4). As such, these fusion proteins can potentiate vaccine-induced immune response or trigger immune responses by themselves. Fc and Fv refer to the constant and variable regions, respectively, of an immunoglobulin molecule, IgG. Nanobody is a single-domain protein derived from the variable regions of Camelidae atypical immunoglobulins.

Peptides and proteins (derived from disease causing microbes or cancerous cells) can be used as antigens in vaccine preparations for infectious disease and cancer. Embodiments of the invention that is disclosed herein can be used to enhance the efficacy of vaccines by using xfcrn-antigen as an antigen-specific adjuvant or antigen delivery vehicle that promotes enhanced antigen-specific T cell responses.

As an example, xfcrn-antigen when used as an antigen delivery vehicle (the peptide or protein antigen in a vaccine is formulated in the form of xfcrn-antigen) the resultant vaccine can be more potent in comparison to a vaccine that employs un-modified antigen (no conjugation to xfcrn).

Vaccine adjuvants that have been developed so far work by: 1) forming depots of antigen at the injection site that leads to slow and extended release of the antigen, 2) forming aggregates of antigen which enables efficient uptake of the antigen by relevant cells of the immune system which in turn increase the proliferation of antigen-specific T cells or 3) stimulating the receptors on certain immune cells (dendritic cells, macrophages, etc) leading to pronounced T cell activation and proliferation. In contrast, the present xfcrn-antigen fusion protein is a novel kind of immune stimulant or adjuvant. Embodiments of the invention relate to the use of antigen delivery vehicles that can be tuned to vary the in vivo persistence of the antigen. One such delivery vehicle, which results in significantly reduced persistence of the antigen and efficient antigen delivery to antigen presenting cells, along with other antigen delivery vehicles with similar functions are being disclosed as inventions herein. xfcrn-antigen works as an adjuvant through a novel mechanism by 1) reducing the in vivo persistence of the antigen and 2) targeting the antigen in an FcRn-dependent fashion (target the antigen to cells expressing FcRn which include many of the relevant immune cells: e.g., antigen presenting cells).

The current invention relates to the use of fusion proteins containing an antigen fused to a protein that can target the neonatal Fc receptor (FcRn) to enhance vaccine-induced antigen-specific CD4+ T cell responses. In some embodiments, one of the antigens used can include an immunodominant peptide (amino acid residues 323-339) derived from chicken egg white ovalbumin [OVA; OVA(323-339)]. In some embodiments, to target FcRn, a mutated Fc portion of mouse immunoglobulin G (mIgG) 1 can be employed. The mutations employed can involve the development of a Fc-mutated human IgG (hIgG) 1 molecule with FcRn-targeting properties [29]. For example, the mutated hIgG1 can include the following amino acid substitutions: Met252Tyr/Ser254Thr/Thr256G1u/His433Lys/Asn434Phe (referred to as ‘MST-HN’). These mutations are localized to the CH2-CH3 region of the Fc and enable MST-HN to bind to FcRn with high affinity at both near neutral and acidic pH. In some embodiments, to impart similar FcRn-targeting properties onto mIgG1 -derived Fc (mIgG1-Fc), amino acid residues 252 (Thr), 256 (Thr), 433 (His) and 434 (Asn) can be mutated to Tyr, Glu, Lys and Phe, respectively. The residue 254 in wild type (WT) mIgG1 is Thr, analogous to that in MST-HN, and hence is not mutated. The mutations introduced into mIgG1-Fc are also localized in the CH2-CH3 region of Fc and are referred to as ‘TT-HN’. hIgG1 (MST-HN) and mIgG1-Fc(TT-HN) can have similar and substantially increased affinities at both acidic and near-neutral pH towards mouse and human FcRn, respectively [10, 29, 70]. As controls, antigen fusions containing mIgG1-Fc(WT) [unmutated] or mIgG1-Fc(H435A) can be generated. H435A refers to a single mutation (His435Ala) in the CH3 region of Fc which abrogates the binding of Fc towards FcRn at both acidic and near-neutral pH. The DNA and amino acid sequences for mIgG1-Fc(WT)-OVA(323-339) (SEQ ID NO:1), mIgG1-Fc(TT-HN)-OVA(323-339) (SEQ ID NO: 2) and mIgG1-Fc(H435A)-OVA(323-339) (SEQ ID NO: 3) are presented below. In addition, as examples, sequences for hIgG1-Fc(MST-HN)-OVA(323-339) (SEQ ID NO: 4), hIgG2-Fc(MST-HN)-OVA(323-339) (SEQ ID NO: 5), hIgG3-Fc(MST-HN)-OVA(323-339) (SEQ ID NO: 6) and hIgG4-Fc(MST-HN)-OVA(323-339) (SEQ ID NO: 7) are also provided below. The amino acid sequences for the hinge, CH2 and CH3 regions of WT hIgG1, hIgG2, hIgG3 and hIgG4 were obtained from the publicly available database, International ImMunoGeneTics Information System [IMGT; Accession numbers—J00228 (hIgG1), J00230 (hIgG2), X03604 (hIgG3) and K01316 (hIgG4)]. In some embodiments, the MST-HN versions of hIgG2, hIgG3 and hIgG4 (or their Fc fragments) can be generated and their binding to FcRn Mutations that enhance binding of hIgG1 towards FcRn can lead to similar effects when introduced into hIgG2, hIgG3 or hIgG4 subtypes [71].

In the sequences presented below, the leader peptide at the N-terminus is included to facilitate the secretion of protein into the culture medium during protein production. The hexapeptide (His6-tag) at the C-terminus is included to enable the purification of the recombinant fusion proteins from cell culture supernatants using Ni2+-NTA-agarose columns. It is reasonable to assume that the function of the recombinant fusion proteins described in this work will be retained by making changes such as: 1) employing an antigenic protein or peptide multimer instead of a single peptide; 2) fusing the antigen at the N-terminus or both N- and C-termini of Fc; 3) modifying the linker composition or length; 4) employing a different FcRn-targeting protein; 5) modifying the dose or time of delivery prior to vaccination; 6) employing a different adjuvant for vaccination instead of complete Freund's adjuvant.

| Sequence 1: Mouse (m)IgG1-Fc(WT)-OVA(323-339) | | |
|---|---|---|
| Marking of the DNA sequence (top) and protein sequence (bottom): | | |
| -N-terminus- | | |
| Leader Peptide - without bolding/italics/underlining | | |
| mIgG1(hinge) - bold | | |
| mIgG1(CH2) - bold + underline | | |
| mIgG1 (CH3) - bold + italics | | |
| Linker(GSGG) and Linker (GSG) - italics | | |
| OVA (323-339) peptide - underlined | | |
| 6 histidines followed by a stop codon - without bolding/italics/underlining | | |
| -C-terminus- | | |
| 1 | ATG GGA TGG AGC TGT ATC ATC CTC TTC TTG GTA GCA ACA GCT ACA | 45 |
| 1 | Met Gly Trp Ser Cys Ile Ile Leu Phe Leu Val Ala Tfr Ala Thr | 15 |
| 46 | FFT FTC CAC TCC GTG CCC AGG GAT TGT GGT TGT AAG CCT TGC ATA | 90 |
| 16 | Gly Val His Ser Val Pro Arg Asp Cys Gly Cys Lys Pro Cys Ile | 30 |
| 91 | TGT ACA GTC CCA GAA GTA TCA TCT GTC TTC ATC TTC CCC CCA AAG | 135 |
| 31 | Cys Thr Val Pro Glu Val Ser Ser Val Phe Ile Phe Pro Pro Lys | 45 |
| 136 | CCC AAG GAT GTG CTC ACC ATT ACT CTG ACT CCT AAG GTC ACG TGT | 180 |
| 46 | Pro Lys Asp Val Leu Thr Ile Tfr Leu Tfr Pro Lys Val Thr Cys | 60 |
| 181 | GTT GTG GTA GAC ATC AGC AAG GAT GAT CCC GAG GTC CAG TTC AGC | 225 |
| 61 | Val Val Val Asp Ile Ser Lys Asp Asp Pro Glu Val Gln Phe Ser | 75 |
| 226 | TGG TTT GTA GAT GAT GTG GAG GTG CAC ACA GCT CAG ACG CAA CCC | 270 |
| 76 | Trp Phe Val Asp Asp Val Glu Val His Thr Ala Gln Thr Gln Pro | 90 |
| 271 | CGG GAG GAG CAG TTC AAC AGC ACT TTC CGC TCA GTC AGT GAA CTT | 315 |
| 91 | Arg Glu Glu Gln Phe Asn Ser Thr Phe Arg Ser Val Ser Glu Leu | 105 |
| 316 | CCC ATC ATG CAC CAG GAC TGG CTC AAT GGC AAG GAG TTC AAA TGC | 360 |
| 106 | Pro Ile Met His Gln Asp Trp Leu Asn Gly Lys Glu Phe Lys Cys | 120 |
| 361 | AGG GTC AAC AGT GCA GCT TTC CCT GCC CCC ATC GAG AAA ACC ATC | 405 |
| 121 | Arg Val Asn Ser Ala Ala Phe Pro Ala Pro Ile Glu Lys Thr Ile | 135 |
| 406 | 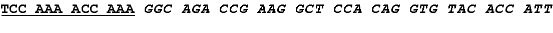 | 450 |
| 136 | 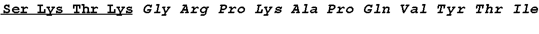 | 150 |
| 451 | 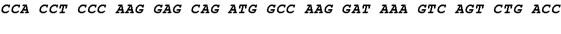 | 495 |
| 151 | 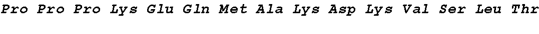 | 165 |
| 496 | 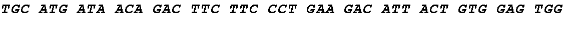 | 540 |
| 166 | 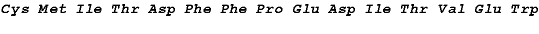 | 180 |
| 541 | 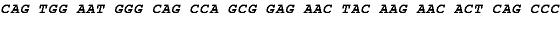 | 585 |
| 181 | 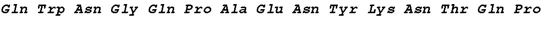 | 195 |
| 586 | 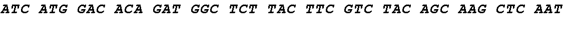 | 630 |
| 196 | 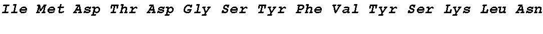 | 210 |
| 631 | 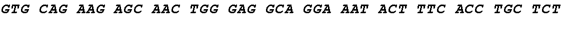 | 675 |
| 211 | 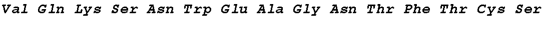 | 225 |
| 676 | 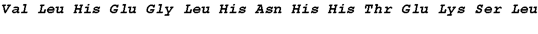 | 720 |
| 226 | 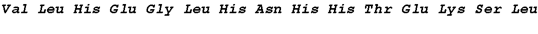 | 240 |
| 721 | 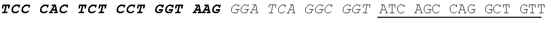 | 765 |
| 241 | 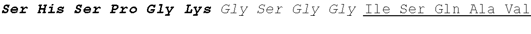 | 255 |
| 766 | CAC GCA GCT CAC GCC GAG ATC AAC GAG GCT GGT AGG GGA TCA GGC | 810 |
| 256 | His Ala Ala His Ala Glu Ile Asn Glu Ala Gly Arg Gly Ser Gly | 270 |
| 811 | CAT CAC CAT CAC CAT CAC TAA | 831 |
| 271 | His His His His His His End | 277 |

| Sequence 2: Mouse (m)IgG1-Fc(TT-HN)-OVA(323-339) | | |
|---|---|---|
| Marking of the DNA sequence (top) and protein sequence (bottom): | | |
| -N-terminus- | | |
| Leader Peptide - without bolding/italics/underlining | | |
| mIgG1(hing) - bold | | |
| mIgG1(CH2) - bold + underline | | |
| mIgG1(CH3) - bold + italics | | |
| 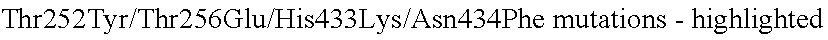 | | |
| Linker(GSGG) and Linker (GSG) - italics | | |
| OVA(323-339) peptide - underlined | | |
| 6 histidines followed by a stop codon - without bolding/italics/underlining | | |
| -C-terminus- | | |
| 1 | ATG GGA TGG AGC TGT ATC ATC CTC TTC TTG GTA GCA ACA GCT ACA | 45 |
| 1 | Met Gly Trp Ser Cys Ile Ile Leu Phe Leu Val Ala Thr Ala Thr | 15 |
| 46 | GGT GTC CAC TCC GTG CCC AGG GAT TGT GGT TGT AAG CCT TGC ATA | 90 |
| 16 | Gly Val His Ser Val Pro Arg Asp Cys Gly Cys Lys Pro Cys Ile | 30 |
| 91 | TGT ACA GTC CCA GAA GTA TCA TCT GTC TTC ATC TTC CCC CCA AAG | 135 |
| 31 | Cys Thr Val Pro Glu Val Ser Ser Val Phe Ile Phe Pro Pro Lys | 45 |
| 136 | 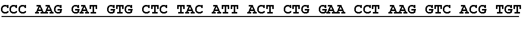 | 180 |
| 46 | 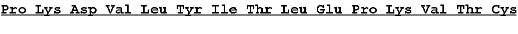 | 60 |
| 181 | GTT GTG GTA GAC ATC AGC AAG GAT GAT CCC GAG GTC CAG TTC AGC | 225 |
| 61 | Val Val Val Asp Ile Ser Lys Asp Asp Pro Glu Val Gln Phe Ser | 75 |
| 226 | TGG TTT GTA GAT GAT GTG GAG GTG CAC ACA GCT CAG ACG CAA CCC | 270 |
| 76 | Trp Phe Val Asp Asp Val Glu Val His Thr Ala Gln Thr Gln Pro | 90 |
| 271 | CGG GAG GAG CAG TTC AAC AGC ACT TTC CGC TCA GTC AGT GAA CTT | 315 |
| 91 | Arg Glu Glu Gln Phe Asn Ser Thr Phe Arg Ser Val Ser Glu Leu | 105 |
| 316 | CCC ATC ATG CAC CAG GAC TGG CTC AAT GGC AAG GAG TTC AAA TGC | 360 |
| 106 | Pro Ile Met His Gln Asp Trp Leu Asn Gly Lys Glu Phe Lys Cys | 120 |
| 361 | AGG GTC AAC AGT GCA GCT TTC CCT GCC CCC ATC GAG AAA ACC ATC | 405 |
| 121 | Arg Val Asn Ser Ala Ala Phe Pro Ala Pro Ile Glu Lys Thr Ile | 135 |
| 406 | 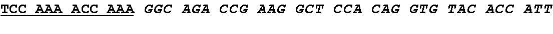 | 450 |
| 136 | 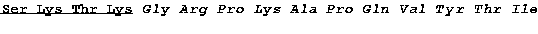 | 150 |
| 451 | 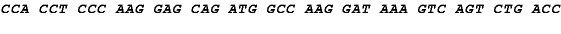 | 495 |
| 151 | 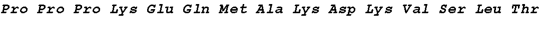 | 165 |
| 496 | 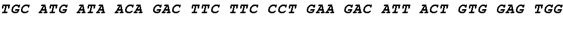 | 540 |
| 166 | 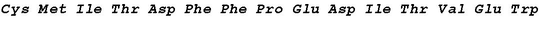 | 180 |
| 541 | 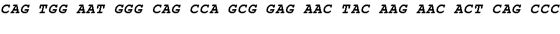 | 585 |
| 181 | 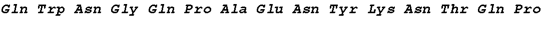 | 195 |
| 586 | 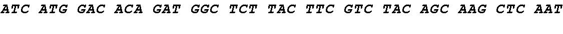 | 630 |
| 196 | 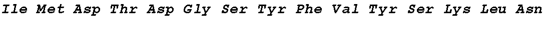 | 210 |
| 631 | 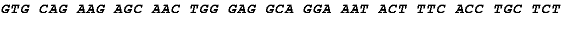 | 675 |
| 211 | 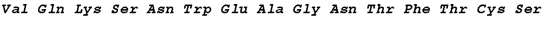 | 225 |
| 676 | 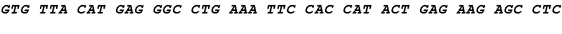 | 720 |
| 226 | 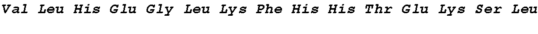 | 240 |
| 721 | 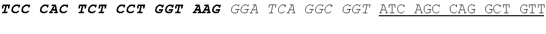 | 765 |
| 241 | 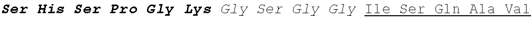 | 255 |
| 766 | CAC GCA GCT CAC GCC GAG ATC AAC GAG GCT GGT AGG GGA TCA GGC | 810 |
| 256 | His Ala Ala His Ala Glu Ile Asn Glu Ala Gly Arg Gly Ser Gly | 270 |
| 811 | CAT CAC CAT CAC CAT CAC TAA | 831 |
| 271 | His His His His His His End | 277 |

| Sequence 3: Mouse(m)IgG1-Fc(H435A)-OVA(323-339) | | |
|---|---|---|
| Marking of the DNA sequence (top) and protein sequence (bottom): | | |
| -N-terminus- | | |
| Leader Peptide - without bolding/italics/underlining | | |
| mIgG1(hinge) - bold | | |
| mIgG1(CH2) - bold + underline | | |
| mIgG1(CH3) - bold + italics | | |
| 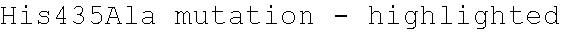 | | |
| Linker (GSGG) and Linker (GSG) - italics | | |
| OVA(323-339) peptide - underlined | | |
| 6 histidines followed by a stop codon - without bolding/italics/underlining | | |
| -C-terminus- | | |
| 1 | ATG GGA TGG AGC TGT ATC ATC CTC TTC TTG GTA GCA ACA GCT ACA | 45 |
| 1 | Met Gly Trp Ser Cys Ile Ile Leu Phe Leu Val Ala Thr Ala Thr | 15 |
| 46 | GGT GTC CAC TCC GTG CCC AGG GAT TGT GGT TGT AAG CCT TGC ATA | 90 |
| 16 | Gly Val His Ser Val Pro Arg Asp Cys Gly Cys Lys Pro Cys Ile | 30 |
| 91 | TGT ACA GTC CCA GAA GTA TCA TCT GTC TTC ATC TTC CCC CCA AAG | 135 |
| 31 | Cys Thr Val Pro Glu Val Ser Ser Val Phe Ile Phe Pro Pro Lys | 45 |
| 136 | CCC AAG GAT GTG CTC ACC ATT ACT CTG ACT CCT AAG GTC ACG TGT | 180 |
| 46 | Pro Lys Asp Val Leu Thr Ile Thr Leu Thr Pro Lys Val Thr Cys | 60 |
| 181 | GTT GTG GTA GAC ATC AGC AAG GAT GAT CCC GAG GTC CAG TTC AGC | 225 |
| 61 | Val Val Val Asp Ile Ser Lys Asp Asp Pro Glu Val Gln Phe Ser | 75 |
| 226 | TGG TTT GTA GAT GAT GTG GAG GTG CAC ACA GCT CAG ACG CAA CCC | 270 |
| 76 | Trp Phe Val Asp Asp Val Glu Val His Thr Ala Gln Thr Gln Pro | 90 |
| 271 | CGG GAG GAG CAG TTC AAC AGC ACT TTC CGC TCA GTC AGT GAA CTT | 315 |
| 91 | Arg Glu Glu Gln Phe Asn Ser Thr Phe Arg Ser Val Ser Glu Leu | 105 |
| 316 | CCC ATC ATG CAC CAG GAC TGG CTC AAT GGC AAG GAG TTC AAA TGC | 360 |
| 106 | Pro Ile Met His Gln Asp Trp Leu Asn Gly Lys Glu Phe Lys Cys | 120 |
| 361 | AGG GTC AAC AGT GCA GCT TTC CCT GCC CCC ATC GAG AAA ACC ATC | 405 |
| 121 | Arg Val Asn Ser Ala Ala Phe Pro Ala Pro Ile Glu Lys Thr Ile | 135 |
| 406 | 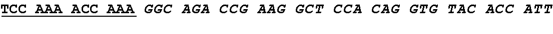 | 450 |
| 136 | 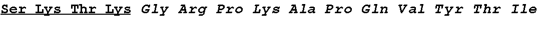 | 150 |
| 451 | 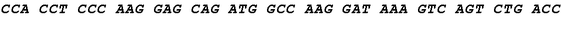 | 495 |
| 151 | 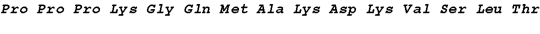 | 165 |
| 496 | 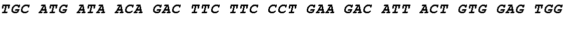 | 540 |
| 166 | 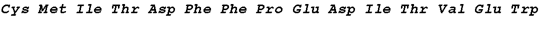 | 180 |
| 541 | 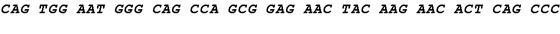 | 585 |
| 181 | 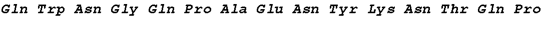 | 195 |
| 586 | 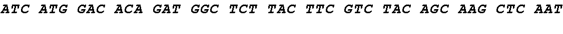 | 630 |
| 196 | 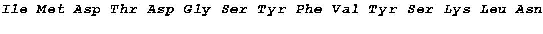 | 210 |
| 631 | 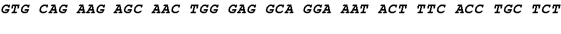 | 675 |
| 211 | 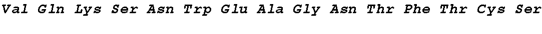 | 225 |
| 676 | 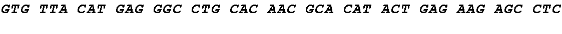 | 720 |
| 226 | 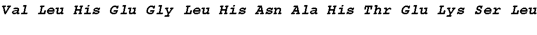 | 240 |
| 721 | 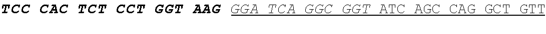 | 765 |
| 241 | 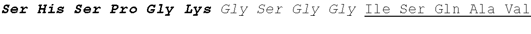 | 255 |
| 766 | CAC GCA GCT CAC GCC GAG ATC AAC GAG GCT GGT AGG GGA TCA GGC | 810 |
| 256 | His Ala Ala His Ala Glu Ile Asn Glu Ala Gly Arg Gly Ser Gly | 270 |
| 811 | CAT CAC CAT CAC CAT CAC TAA | 831 |
| 271 | His His His His His His End | 277 |

| Sequence 4: Human (h)IgG1-Fc(MST-HN)-OVA(323-339) | | |
|---|---|---|
| Marking of the DNA sequence (top) and protein sequence (bottom): | | |
| -N-terminus- | | |
| Leader Peptide - without bolding/italics/underlining | | |
| hIgG1(hinge) - bold | | |
| hIgG1(CH2) - bold + underline | | |
| hIgG1(CH3) - bold + italics | | |
| 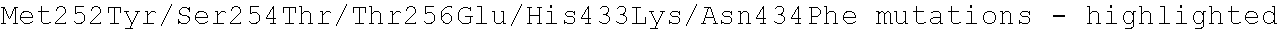 | | |
| Linker (GSGG) and Linker (GSG) - italics | | |
| OVA(323-339) peptide - underlined | | |
| 6 histidines followed by a stop codon - without bolding/italics/underlining | | |
| -C-terminus- | | |
| 1 | ATG GGA TGG AGC TGT TATC ATC CTC TTC TTG GTA GCA ACA GCT ACA | 45 |
| 1 | Met Gly Trp Ser Cys Ile Ile Leu Phe Leu Val Ala Thr Ala Thr | 15 |
| 46 | GGT GTC CAC TCC GAG CCC AAG AGC TGC GAC AAG ACC CAC ACC TGC | 90 |
| 16 | Gly Val His Ser Glu Pro Lys Ser Cys Asp Lys Thr His Htr Cys | 30 |
| 91 | CCC CCC TGC CCC GCC CCC GAG CTG CTG GGC GGC CCC AGC GTG TTC | 135 |
| 31 | Pro Pro Cys Pro Ala Pro Glu Leu Leu Gly Gly Pro Ser Val Phe | 45 |
| 136 | 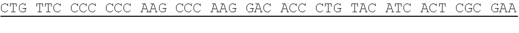 | 180 |
| 46 | 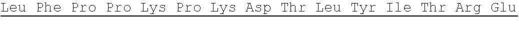 | 60 |
| 181 | CCC GAG GTG ACC TGC GTG GTG GTG GAC GTG AGC CAC GAG GAC CCC | 225 |
| 61 | Pro Glu Val Thr Cys Val Val Val Asp Val Ser His Glu Asp Pro | 75 |
| 226 | GAG GTG AAG TTC AAC TGG TAC GTG GAC GGC GTG GAG GTG CAC AAC | 270 |
| 76 | Glu Val Lys Phe Asn Trp Tyr Val Asp Gly Val Glu Val His Asn | 90 |
| 271 | GCC AAG ACC AAG CCC CGC GAG GAG CAG TAC AAC AGC ACC TAC CGC | 315 |
| 91 | Ala Lys Thr Lys Pro Arg Glu Glu Gln Tyr Asn Ser Thr Tyr Arg | 105 |
| 316 | GTG GTG AGC GTG CTG ACC GTG CTG CAC CAG GAC TGG CTG AAC GGC | 360 |
| 106 | Val Val Ser Val Leu Thr Val Leu His Gln Asp Trp Leu Asn Gly | 120 |
| 361 | AAG GAG TAC AAG TGC AAG GTG AGC AAC AAG GCC CTG CCC GCC CCC | 405 |
| 121 | Lys Glu Tyr Lys Cys Lys Val Ser Asn Lys Ala Leu Pro Ala Pro | 135 |
| 406 | 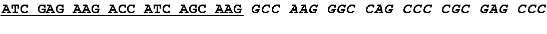 | 450 |
| 136 | 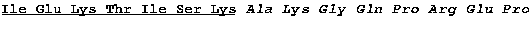 | 150 |
| 451 | 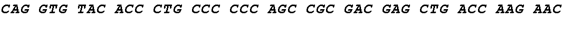 | 495 |
| 151 | 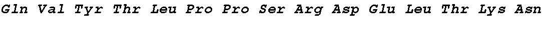 | 165 |
| 496 | 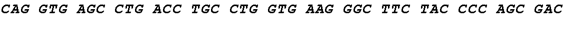 | 540 |
| 166 | 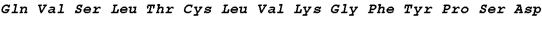 | 180 |
| 541 | 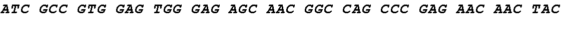 | 585 |
| 181 | 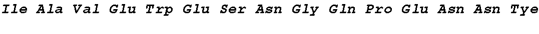 | 195 |
| 586 | 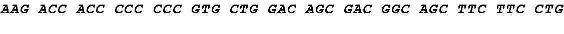 | 630 |
| 196 | 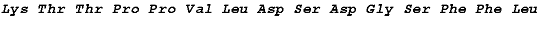 | 210 |
| 631 | 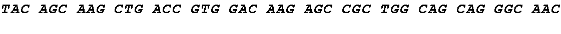 | 675 |
| 211 | 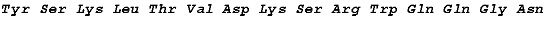 | 225 |
| 676 | 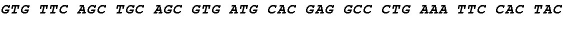 | 720 |
| 226 | 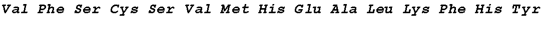 | 240 |
| 721 | 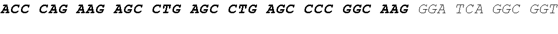 | 765 |
| 241 | 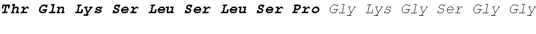 | 255 |
| 766 | ATC AGC CAG GCT GTT CAC GCA GCT CAC GCC GAG ATC AAC GAG GCT | 810 |
| 256 | Ile Ser Gln Ala Val His Ala Ala His Ala Glu Ile Asn Glu Ala | 270 |
| 811 | GGT AGG GGA TCA GGC CAT CAC CAT CAC CAT CAC TAA | 846 |
| 271 | Gly Arg Gly Ser Gly His His His His His His End | 282 |

| Sequence 5: Human (h)IgG2-Fc(MST-HN)-OVA(323-339) | | |
|---|---|---|
| Marking of the DNA sequence (top) and protein sequence (bottom): | | |
| -N-terminus- | | |
| Leader Peptide - without bolding/italics/underlining | | |
| hIgG2(hinge) - bold | | |
| hIgG2(CH2) - bold + underline | | |
| hIgG2(CH3) - bold + italics | | |
| 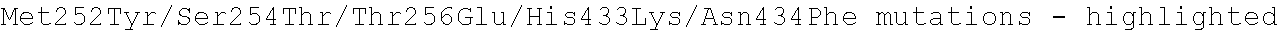 | | |
| Linker (GSGG) and Linker (GSG) - italics | | |
| OVA(323-339) peptide - underlined | | |
| 6 histidines followed by a stop codon - without bolding/italics/underlining | | |
| -C-terminus- | | |
| 1 | ATG GGA TGG AGC TGT ATC ATC CTC TTC TTG GTA GCA ACA GCT ACA | 45 |
| 1 | Met Gly Trp Ser Cys Ile Ile Leu Phe Leu Val Ala Thr Ala Thr | 15 |
| 46 | GGT GTC CAC TCC GAG CGC AAG TGC TGC GTG GAG TGC CCC CCC TGC | 90 |
| 16 | Gly Val His Ser Glu Arg Lys Cys Cys Val Glu Cys Pro Pro Cys | 30 |
| 91 | CCC GCC CCC CCC GTG GCC GGC CCC AGC GTG TTC CTG TTC CCC CCC | 135 |
| 31 | Pro Ala Pro Pro Val Ala Gly Pro Ser Val Phe Leu Phe Pro Pro | 45 |
| 136 | 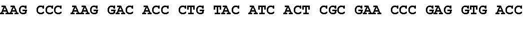 | 180 |
| 46 | 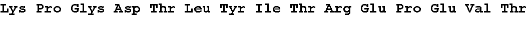 | 60 |
| 181 | TGC GTG GTG GTG GAC GTG AGC CAC GAG GAC CCC GAG GTG CAG TTC | 225 |
| 61 | Cys Val Val Val Asp Val Ser His Glu Asp Pro Glu Val Gln Phe | 75 |
| 226 | AAC TGG TAC GTG GAC GGC GTG GAG GTG CAC AAC GCC AAG ACC AAG | 270 |
| 76 | Asn Trp Tyr Val Asp GBly Val Glu Val His Asn Ala Lys Thr Lys | 90 |
| 271 | CCC CGC GAG GAG CAG TTC AAC AGC ACC TTC CGC GTG GTG AGC GTG | 315 |
| 91 | Pro Arg Glu Glu Gln Phe Asn Ser Thr Phe Arg Val Val Ser Val | 105 |
| 316 | CTG ACC GTG GTG CAC CAG GAC TGG CTG AAC GGC AAG GAG TAC AAG | 360 |
| 106 | Leu Thr Val Val His Gln Asp Trp Leu Asn Gly Lys Glu Tyr Lys | 120 |
| 361 | TGC AAG GTG AGC AAC AAG GGC CTG CCC GCC CCC ATC GAG AAG ACC | 405 |
| 121 | Cys Lys Val Ser Asn Lys Gly Leu Pro Ala Pro Ile Glu Lys Thr | 135 |
| 406 | 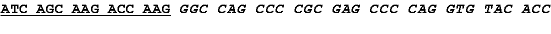 | 450 |
| 136 | 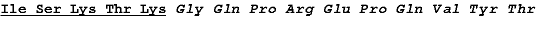 | 150 |
| 451 | 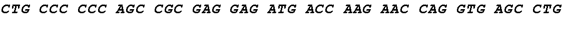 | 495 |
| 151 | 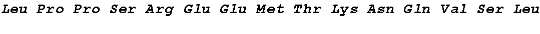 | 165 |
| 496 | 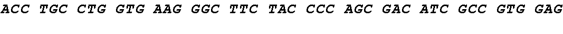 | 540 |
| 166 | 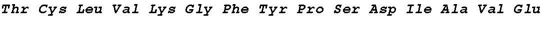 | 180 |
| 541 | 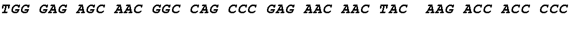 | 585 |
| 181 | 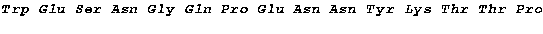 | 195 |
| 586 | 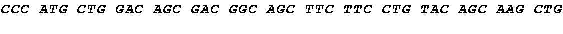 | 630 |
| 196 | 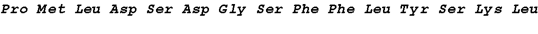 | 210 |
| 631 | 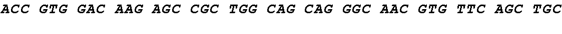 | 675 |
| 211 | 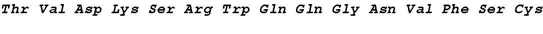 | 225 |
| 676 | 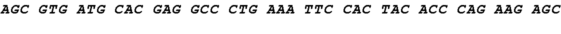 | 720 |
| 226 | 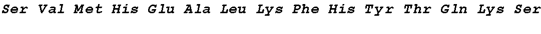 | 240 |
| 721 | 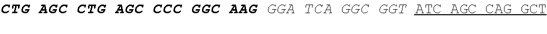 | 765 |
| 241 | 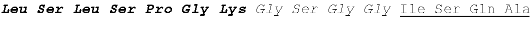 | 255 |
| 766 | GTT CAC GCA GCT CAC GCC GAG ATC AAC GAG GCT GGT AGG GGA TCA | 810 |
| 256 | Val His Ala Ala His Ala Glu Ile Asn Glu Ala Gly Arg Gly Ser | 270 |
| 811 | GGC CAT CAC CAT CAC CAT CAC TAA | 834 |
| 271 | Gly His His His His His His End | 278 |

| Sequence 6: Human (h)IgG3-Fc(MST-HN)-OVA(323-339) | | |
|---|---|---|
| Marking of the DNA sequence (top) and protein sequence (bottom): | | |
| -N-terminus- | | |
| Leader Peptide - without bolding/italics/underlining | | |
| hIgG3(hinge) - bold | | |
| hIgG3(CH2) - bold + underline | | |
| hIgG3(CH3) - bold + italics | | |
| 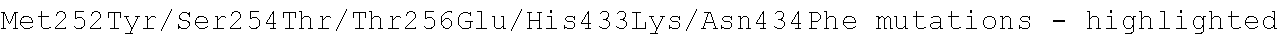 | | |
| Linker (GSGG) and Linker (GSG) - italics | | |
| OVA(323-339) peptide - underlined | | |
| 6 histidines followed by a stop codon - without bolding /italics/underlining | | |
| -C-terminus- | | |
| 1 | ATG GGA TGG AGC TGT ATC ATC CTC TTC TTG GTA GCA ACA GCT ACA | 45 |
| 1 | Met Gly Trp Ser Cys Ile Ile Leu Phe Leu Val Ala Thr Ala Thr | 15 |
| 46 | GGT GTC CAC TCC GAG CTG AAG ACC CCC CTG GGC GAC ACC ACC CAC | 90 |
| 16 | Gly Val His Ser Glu Leu Lys Thr Pro Leu Gly Asp Thr Thr His | 30 |
| 91 | ACC TGC CCC CGC TGC CCC GAG CCC AAG AGC TGC GAC ACC CCC CCC | 135 |
| 31 | Thr Cys Pro Arg Cys Pro Glu Pro Lys Ser Cys Asp Thr Pro Pro | 45 |
| 136 | CCC TGC CCC CGC TGC CCC GAG CCC AAG AGC TGC GAC ACC CCC CCC | 180 |
| 46 | Pro Cys Pro Arg Cys Pro Glu Pro Lys Ser Cys Asp Thr Pro Pro | 60 |
| 181 | CCC TGC CCC CGC TGC CCC GAG CCC AAG AGC TGC GAC ACC CCC CCC | 225 |
| 61 | Pro Cys Pro Arg Cys Pro Glu Pro Lys Ser Cys Asp Thr Pro Pro | 75 |
| 226 | CCC TGC CCC CGC TGC CCC GCC CCC GAG CTG CTG GGC GGC CCC AGC | 270 |
| 76 | Pro Cys Pro Arg Cys Pro Ala Pro Glu Leu Leu Gly Gly Pro Ser | 90 |
| 271 | 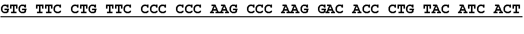 | 315 |
| 91 | 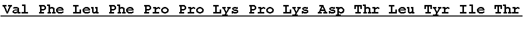 | 105 |
| 316 | 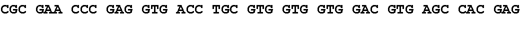 | 360 |
| 106 | 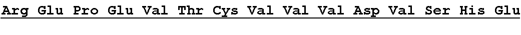 | 120 |
| 361 | GAC CCC GAG GTG CAG TTC AAG TGG TAC GTG GAC GGC GTG GAG GTG | 405 |
| 121 | Asp Pro Glu Val Gln Phe Lys Trp Tyr Val Asp Gly Val Glu Val | 135 |
| 406 | CAC AAC GCC AAG ACC AAG CCC CGC GAG GAG CAG TAC AAC AGC ACC | 450 |
| 136 | His Asn Ala Lys Thr Lys Pro Arg Glu Glu Gln Tyr Asn Ser Thr | 150 |
| 451 | TTC CGC GTG GTG AGC GTG CTG ACC GTG CTG CAC CAG GAC TGG CTG | 495 |
| 151 | Phe Arg Val Val Ser Val Leu Thr Val Leu His Gln Asp Trp Leu | 165 |
| 496 | AAC GGC AAG GAG TAC AAG TGC AAG GTG AGC AAC AAG GCC CTG CCC | 540 |
| 166 | Asn Gly Lys Glu Tyr Lys Cys Lys Val Ser Asn Lys Ala Leu Pro | 180 |
| 541 | 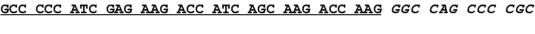 | 585 |
| 181 | 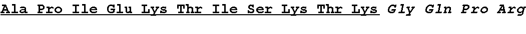 | 195 |
| 586 | 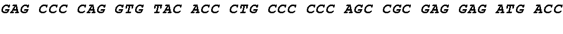 | 630 |
| 196 | 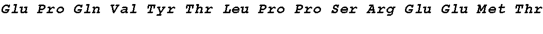 | 210 |
| 631 | 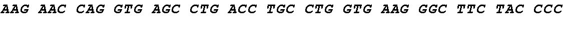 | 675 |
| 211 | 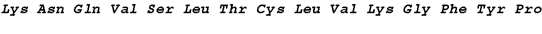 | 225 |
| 676 | 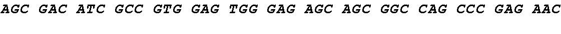 | 720 |
| 226 | 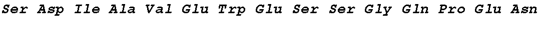 | 240 |
| 721 | 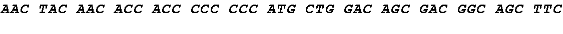 | 765 |
| 241 | 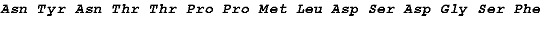 | 255 |
| 766 | 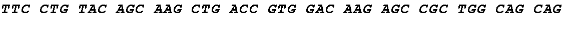 | 810 |
| 256 | 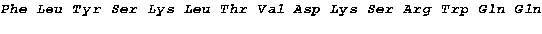 | 270 |
| 811 | 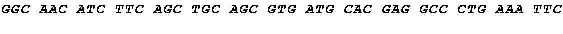 | 855 |
| 271 | 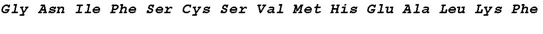 | 285 |
| 856 | 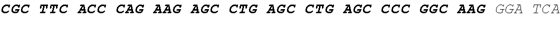 | 900 |
| 286 | 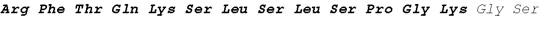 | 300 |
| 901 | GGC GGT ATC AGC CAG GCT GTT CAC GCA GCT CAC GCC GAG ATC AAC | 945 |
| 301 | Gly Gly Ile Ser Gln Ala Val His Ala Ala His Ala Glu Ile Asn | 315 |
| 946 | GAG GCT GGT AGG GGA TCA GGC CAT CAC CAT CAC CAT CAC TAA | 987 |
| 316 | Glu Ala Gly Arg Gly Ser Gly His His His His His His End | 329 |

| Sequence 7: Human (h)IgG4-Fc(MST-HN)-OVA(323-339) | | |
|---|---|---|
| Marking of the DNA sequence (top) and protein sequence (bottom): | | |
| -N-terminus- | | |
| Leader Peptide - without bolding/italics/underlining | | |
| hIgG4(hinge) - bold | | |
| hIgG4(CH2) - bold + underline | | |
| hIgG4(CH3) - bold + italics | | |
| 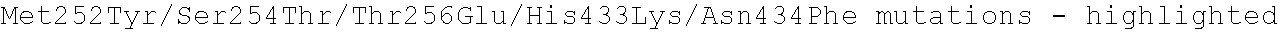 | | |
| Linker (GSGG) and Linker (GSG) - italics | | |
| OVA(323-339) peptide - underlined | | |
| 6 histidines followed by a stop codon - without bolding/italics/underlining | | |
| -C-terminus- | | |
| 1 | ATG GGA TGG AGC TGT ATC ATC CTC TTC TTG GTA GCA ACA GCT ACA | 45 |
| 1 | Met Gly Trp Ser Cys Ile Ile Leu Phe Leu Val Ala Thr Ala Thr | 15 |
| 46 | GGT GTC CAC TCC GAG AGC AAG TAC GGC CCC CCC TGC CCC AGC TGC | 90 |
| 16 | Gly Val His Ser Glu Ser Lys Tyr Gly Pro Por Cys Pro Ser Cys | 30 |
| 91 | CCC GCC CCC GAG TTC CTG GGC GGC CCC AGC GTG TTC CTG TTC CCC | 135 |
| 31 | Pro Ala Pro Glu Phe Leu Gly Gly Pro Ser Val Phe Leu Phe Pro | 45 |
| 136 | 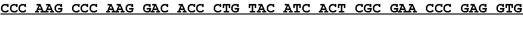 | 180 |
| 46 | 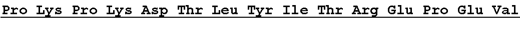 | 60 |
| 181 | ACC TGC GTG GTG GTG GAC GTG AGC CAG GAG GAC CCC GAG GTG CAG | 225 |
| 61 | Thr Cys Val Val Val Asp Val Ser Gln Glu Asp Pro Glu Val Gln | 75 |
| 226 | TTC AAC TGG TAC GTG GAC GGC GTG GAG GTG CAC AAC GCC AAG ACC | 270 |
| 76 | Phe Asn Trp Tyr Val Asp Gly Val Glu Val His Asn Ala Lys Thr | 90 |
| 271 | AAG CCC CGC GAG GAG CAG TTC AAC AGC ACC TAC CGC GTG GTG AGC | 315 |
| 91 | Lys Pro Arg Glu Glu Gln Phe Asn Ser Thr Tyr Arg Val Val Ser | 105 |
| 316 | GTG CTG ACC GTG CTG CAC CAG GAC TGG CTG AAC GGC AAG GAG TAC | 360 |
| 106 | Val Leu Thr Val Leu His Gln Asp Trp Leu Asn Gly Lys Glu Tyr | 120 |
| 361 | AAG TGC AAG GTG AGC AAC AAG GGC CTG CCC AGC AGC ATC GAG AAG | 405 |
| 121 | Lys Cys Lys Val Ser Asn Lys Gly Leu Pro Ser Ser Ile Glu Lys | 135 |
| 406 | 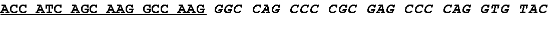 | 450 |
| 136 | 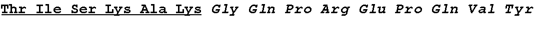 | 150 |
| 451 | 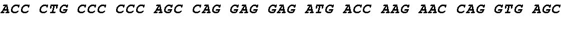 | 495 |
| 151 | 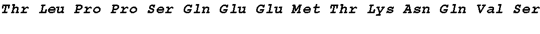 | 165 |
| 496 | 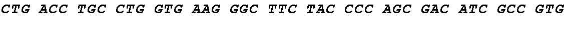 | 540 |
| 166 | 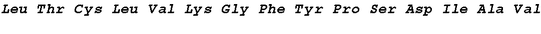 | 180 |
| 541 | 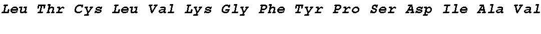 | 585 |
| 181 | 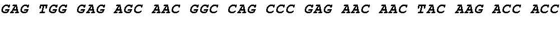 | 195 |
| 586 | 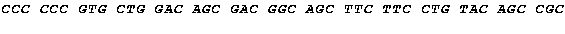 | 630 |
| 196 | 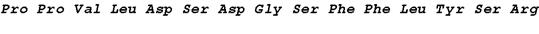 | 210 |
| 631 | 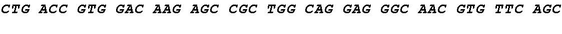 | 675 |
| 211 | 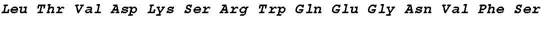 | 225 |
| 676 | 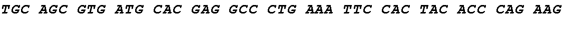 | 720 |
| 226 | 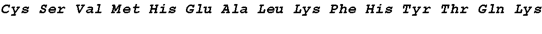 | 240 |
| 721 | 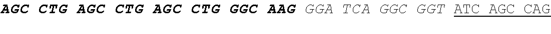 | 765 |
| 241 | 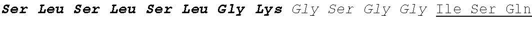 | 255 |
| 766 | GCT GTT CAC GCA GCT CAC GCC GAG ATC AAC GAG GCT GGT AGG GGA | 810 |
| 256 | Ala Val His Ala Ala His Ala Glu Ile Asn Glu Ala Gly Arg Gly | 270 |
| 811 | TCA GGC CAT CAC CAT CAC CAT CAC TAA | 837 |
| 271 | Ser Gly His His His His His His End | 279 |

Embodiments of the invention can include sequences, constructs, and fusions employing mammalian sequences, mouse sequences, humanized non-human mammalian sequences such as, for example, humanized mouse sequences, as well as human sequences, for the relevant regions. Likewise, embodiments of the invention can include sequences, constructs, and fusions employing engineered and/or designed and/or combined segments of natural sequences of variants of human sequences to account for variants in the human population at the relevant positions of the genome. The design of such humanized sequences, variants, and the like are within the knowledge of those of ordinary skill in the art.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Fusions Comprising Recombinant Fc Fragments Fused to Myelin Basic Protein Peptide—Materials and Methods for Examples 2-8

### Mice

B10.PL (H-2^{u}) mice were purchased from the Jackson Laboratory (Bar Harbor, Me.). Mice that transgenically express the 1934.4 TCR (1934.4 tg mice [18]) or clone 19 TCR (T/R⁺ tg mice [19]) were kindly provided by Dr. Hugh McDevitt (Stanford University, Calif.) and Dr. Juan Lafaille (New York University School of Medicine, N.Y.), respectively. Both the 1934.4 and clone 19 TCRs are specific for MBP1-9 complexed with I-A^{u} [18, 19] and have similar affinities for antigen [20]. Mice were bred in a specific pathogen-free facility at the University of Texas Southwestern Medical Center or Texas A&M University and were handled in compliance with institutional policies and protocols approved by the Institutional Animal Care and Use Committees. 6-10 week old male or female mice were used in experiments.

### Peptides

The N-terminal, acetylated peptide of MBP (MBP1-9, Ac-ASQKRPSQR) and MBP1-9(4Y) (Ac-ASQYRPSQR) were purchased from CS Bio (Menlo Park, Calif.).

### Production of Recombinant Proteins

Expression constructs for the production of full length anti-lysozyme antibodies (WT, m-set-1 and m-set-2) were generated by isolating the cDNA encoding the heavy chain and light chain from the D1.3 hybridoma (mouse IgG1, anti-hen egg lysozyme) [21]. The mutations were inserted into the WT heavy chain gene using splicing by overlap extension and cloned into pOptiVEC™-TOPO® vector (Life Technologies, Grand Island, N.Y.) for expression. The light chain gene was cloned into pcDNA™ 3.3-TOPO® vector (Life Technologies, Grand Island, N.Y.). Complete sequences of expression plasmids are available upon request. The light chain expression construct was transfected into CHO DG44 cells by electroporation. Stable clones of CHO DG44 cells were selected for light chain expression using previously described methods [22]. The light chain transfectant expressing the highest levels of recombinant protein was used as a recipient for the heavy chain constructs. Clones expressing the highest levels of anti-lysozyme antibody were selected and recombinant antibodies purified from culture supernatants using lysozyme-Sepharose [23]. Mouse IgG1 (anti-hen egg lysozyme, D1.3 [21]) was purified using lysozyme-Sepharose [23] from hybridoma culture supernatants.

Expression plasmids encoding WT or mutated (m-set-2) mouse IgG1-derived Fc-hinge connected at the C-termini through a Gly-Ser-Gly-Gly linker to codons encoding the MBP1-9(4Y) epitope or MBP1-9(4Y) epitope with residues 3 and 6 of the peptide replaced by alanine have been described previously [10]. The glycine at the N-terminus of the peptide mimics the acetyl group that is necessary for T cell recognition of the MBP epitope [24]. The m-set-1 mutations were inserted into the WT Fc-MBP fusion construct using splicing by overlap extension and designed oligonucleotide primers. All Fc-MBP fusion genes were cloned into pEF6/V5-His vector (Life Technologies, Grand Island, N.Y.). Fc-MBP fusion constructs were transfected into CHO-S cells, stable transfectants selected and recombinant proteins purified from culture supernatants as described previously [10]. Analogous methods were used to generate Fc-hinge variants (WT, m-set-1, m-set-2) without the C-terminal MBP1-9 epitope. Complete sequences of expression constructs are available upon request.

### Recombinant Peptide-MHC Complexes

Soluble, recombinant MBP1-9(4Y):I-A^{u} complexes were generated using baculovirus-infected High Five insect cells and purified as described previously [25]. The complexes were site-specifically biotinylated and multimeric complexes (“tetramers”) were generated using PE-labeled ExtrAvidin (Sigma-Aldrich, St. Louis, MO).

### Cell Lines

The MBP1-91-A^{u}-specific T cell hybridoma #46 has been described previously [26]. The I-A^{u}-expressing B lymphoblastoid line PL8 was generously provided by Dr. David Wraith (University of Bristol, Bristol, U.K.). PL8:FcRn cells were generated by stably transfecting PL8 cells with an expression construct encoding mouse FcRn tagged at the C-terminus with GFP, followed by selection with G418 (600 μg/ml, Life Technologies, Grand Island, N.Y.) [10].

### Surface Plasmon Resonance Analyses

Equilibrium dissociation constants of WT and mutated mouse Fc-hinge fragments (IgG1-derived) for binding to recombinant mouse FcRn were determined using surface plasmon resonance and a BlAcore 2000. Mouse Fc-hinge fragments were immobilized by amine coupling chemistry (to a density of ˜250-850 RU) and BlAcore experiments carried out as described previously, using soluble mouse FcRn in Dulbecco's phosphate-buffered saline (DPBS) plus 0.01% Tween pH 6.0 or 7.4 as analyte [27]. FcRn binds to two sites on IgG that are not equivalent [27]. This results in K_{D} estimates for two dissociation constants, and the values for the higher affinity interaction sites are presented. The data were processed as described previously [27].

### T Cell Stimulation Assay

Fc-MBP fusions were added to 96-well plates containing PL-8 or PL-8:FcRn cells (5×10⁴ cells/well) and MBP1-9:I-A^{u}-specific T cell hybridoma #46 cells (5×10⁴ cells/well). IL-2 levels in culture supernatants following 24 hours of incubation were assessed using a sandwich ELISA with the following reagents: rat anti-mouse IL-2 capture antibody (clone, JES6-1A12; Becton-Dickinson, San Jose, Calif.), biotinylated rat anti-mouse IL-2 detection antibody (clone, JES6-5H4; Becton-Dickinson, San Jose, Calif.) and ExtrAvidin-Peroxidase (Sigma-Aldrich, St. Louis, Mo).

### Pharmacokinetic Experiments

6-10 week old female B10.PL mice were fed 0.1% Lugol (Sigma-Aldrich, St. Louis, Mo.) in water for 72 h before i.v. injection in the tail vein with ¹²⁵I-labeled IgGs or Fc-MBP fusions (10-15 μg per mouse). Levels of radioactivity in 10 μl blood samples were determined at the indicated times by gamma counting. To determine the AUC for IgGs and Fc-MBP fusion proteins, data were fitted to a bi-exponential decay model using custom software written in MATLAB (Mathworks, Natick, Mass.). The area under each of these bi-exponential model curves between time t=0 and the time at which the extrapolated curve reaches 1% of the injected dose was calculated.

To investigate whether the Fc-MBP fusions affected the activity of FcRn in regulating the clearance rate of IgG, 6-10 week old male B10.PL mice were fed 0.1% Lugol (Sigma-Aldrich, St. Louis, Mo.) in drinking water for 72 h prior to i.v. injection with 10-15 μg ¹²⁵I-labeled mouse IgG1 (anti-hen egg lysozyme, D1.3). 24 hours later, the mice were i.v. injected with 1 μg Fc-MBP fusion or vehicle (DPBS) control. Levels of radioactivity in 10 μl blood samples were analyzed at the indicated times by gamma counting and β-phase half-lives following injection of Fc-MBP fusion or vehicle determined as described previously [28].

### Analyses of Proliferative Responses of Transferred Antigen-Specific T Cells

Antigen-specific CD4⁺ T cells were isolated from the splenocytes of MBP1-9:I-A^{u}-specific TCR transgenic mice (1934.4 tg [18] and T/R⁻ tg [19]) through negative selection using a MACS CD4⁺ T cell isolation kit (Miltenyi Biotec, San Diego, Calif.). Female B10.PL mice were i.v. injected with 1 μg Fc-MBP fusion. One hour (Day 0′), 3 or 5 days following Fc-MBP fusion delivery, 5×10⁵ CFSE-labeled CD4⁺ T cells were injected i.v. into the mice. Three days later, splenocytes and LN cells were isolated for flow cytometry analyses.

### Induction of EAE

8-10 week old male B10.PL mice were immunized subcutaneously at four sites in the flanks with 200 μg acetylated MBP1-9 (CS Bio, Menlo Park, Calif.) emulsified with complete Freund's adjuvant (Sigma Aldrich, St. Louis, Mo.) containing an additional 4 mg/ml heat-inactivated Mycobacterium tuberculosis (strain H37Ra, Becton-Dickinson, San Jose, Calif.). In addition, 200 ng pertussis toxin (List Biological Laboratories, Campbell, Calif.) was injected i.p. on days 0 (0 h) and 2 (45 h).

Scoring of disease activity was as follows: 0, no paralysis; 1, limp tail; 2, moderate hind limb weakness; 3, severe hind limb weakness; 4, complete hind limb paralysis; 5, quadriplegia; and 6, death due to disease. Clinical signs of EAE were assessed for up to 30 days after immunization.

Prophylactic and Therapeutic Treatment of Mice with Fc-MBP Fusions

For tolerance induction in a prophylactic setting, male B10.PL mice were injected i.v. with 1 μg Fc-MBP fusion and seven days later, immunized with MBP1-9 and treated with pertussis toxin to induce EAE. In some experiments, mice were treated with 5 doses of 1 μg Fc(v.short)-MBP (starting at 7 days prior to immunization, at 36 hour intervals) or with a single dose of 5 μg Fc(v.short)-MBP delivered 7 days prior to immunization. For tolerance induction during ongoing disease, mice were injected i.v. with 1 μg Fc-MBP fusion at the onset of EAE (mean clinical score of 1-2).

### Antibodies and Flow Cytometry Analyses

Single cell suspensions from spleen, draining LNs (axillary, brachial and inguinal), brain and spinal cord were obtained by mechanical disruption and forcing through 70 μm cell strainers (Becton-Dickinson, San Jose, Calif.). For experiments involving analyses of immune cells in the CNS, mice were perfused with heparinized DPBS before collecting the organs. Splenic cell suspensions were depleted of erythrocytes using red blood cell lysis buffer.

Mononuclear cells from CNS cell suspensions were obtained using Percoll (1131 g/ml, GE Healthcare) gradients. Briefly, cells were washed with 37% Percoll and suspended in 30% Percoll which was then layered over 70% Percoll and centrifuged at 2118 g. Following centrifugation, the cells at the interface were collected, washed with DPBS and used for flow cytometry analyses.

For intracellular staining to detect Foxp3 and T-bet, cells were initially surface-stained, followed by fixation and permeabilization using Foxp3 staining buffer set (eBioscience, San Diego, Calif.). Permeabilized cells were incubated with fluorescently labeled anti-Foxp3 or anti-T-bet antibodies and washed with DPBS.

To detect antigen-specific CD4⁺ T cells, single cell suspensions from spleens and LNs were incubated with PE-labeled MBP1-9(4Y):I-Atetramers for 90 min at 12° C., followed by washing with DPBS.

Flow cytometry analyses were performed using a FACSCalibur (Becton-Dickinson, San Jose, Calif.) or LSRFortessa (Becton-Dickinson, San Jose, Calif.) and data analyzed using FlowJo (Tree Star, Ashland, Oreg.). Antibodies specific for the following were purchased from either Becton-Dickinson (San Jose, Calif.), eBioscience (San Diego, Calif.) or Biolegend (San Diego, Calif): CD4 (RM4-5), Foxp3 (FJK-16s), T-bet (4B10), CD4OL (MR1), F4/80 (BM8), PD-1 (29F.1Al2), CTLA-4 (UC10-4B9), LFA-1 (H155-78), CXCR3 (CXCR3-173), α4 (R1-2), β1 (HMβ1-1), α4β7(DATK32) and CD45 (30-F11).

### Statistical Analyses

Tests for statistical significance for flow cytometric analyses of cell numbers and pharmacokinetic data were carried out using two-tailed Student's t-test in the statistics toolbox of MATLAB (Mathworks, Natick, Mass.). Due to the longitudinal nature of the measures of clinical scores over time, we compared the clinical score profiles between the groups of mice in disease experiments using the linear mixed effects model with AR(1) covariance structure with Statistical Analysis System software (SAS Institute Inc., Cary, N.C.). p values of less than 0.05 were taken to be significant.

### Example 2

### Generation of Fc-Antigen Fusion Proteins with Different in Vivo Dynamics

The binding of WT mouse IgG1 or corresponding Fc fragment to mouse FcRn is highly pH-dependent, with binding at pH 5.5-6 (early-late endosomes) that becomes negligible at pH 7-7.4 [11]. Engineered IgGs with higher binding affinity than WT IgG1 for FcRn at both acidic and near-neutral pH confers increased (receptor-mediated) uptake of the antibody, limited exocytic release during recycling, entry into lysosomes and reduced persistence [28, 29]. Two sets of Fc mutations that alter FcRn binding were selected for this study: mutation-set (m-set)-1 (T252L/T254S/T256F/E380A/H433K/N434F) [30-32] and m-set-2 (T252Y/T256E/H433K/N434F) [29]. Based on the effects of these mutations on the equilibrium dissociation constants (K_{D}s) of the interactions of mouse IgG1-derived Fc-hinge fragments with mouse FcRn (Table 1), Fc fragments or IgG molecules harboring m-set-1 and m-set-2 mutations would be predicted to have distinct dynamic properties in vivo [11].

TABLE 1
Binding properties of mouse Fc fragments

Binding to FcRn
(K_{D}, nM)

| | pH 6.0 | pH 7.4 |
|---|---|---|
| WT | 218.2 | N.B.* |
| m-set-1 | 2.6 | 114.6 |
| m-set-2 | 1.1 | 20.4 |

*N.B. = no detectable binding.

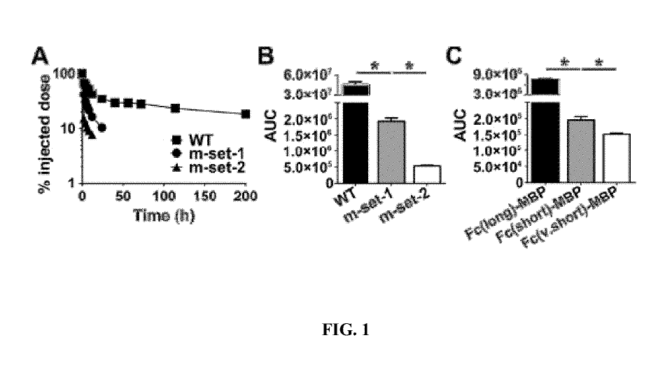

Fc-MBP fusions comprising WT or mutated Fc fragments linked to MBP1-9 were generated. Although multiple studies have demonstrated that this MBP peptide requires N-terminal acetylation for T cell recognition, the replacement of the acetyl group with glycine generates an analogous epitope [24]. Further, the fusion proteins contain the ‘4Y’ analog [MBP1-9(4Y)] of this peptide, in which lysine at position 4 is substituted by tyrosine. This analog has higher binding affinity for I-A^{u} than its parent peptide whilst retaining recognition by autoreactive T cells [24, 33]. The pharmacokinetics of the Fc-MBP fusions were analyzed in mice (FIG. 1C). Despite the lower persistence of the Fc fusions compared with the corresponding parent IgGs, most likely due to the binding of the epitope extending from the CH3 domain of the Fc fragment to the MHC Class II molecule, I-A^{u} [34], the in vivo exposure (AUC) to the proteins decreased in the same order (FIG. 1C). Throughout these studies, fusion proteins containing WT or Fc fragments with m-set-1 and m-set-2 mutations were therefore designated Fc(long)-MBP, Fc(short)-MBP and Fc(v.short)-MBP, respectively. Although the difference in exposure (AUC) between Fc(short)-MBP and Fc(v.short)-MBP was significant, this difference was much lower than that for Fc(short)-MBP compared with Fc(long)-MBP (FIG. 1C). Consistent with the differences in exposure for the Fc-MBP fusions, the percentage remaining of the injected dose after one hour was 16.33±0.63% and 9.62±0.28% for Fc(short)-MBP and Fc(v.short)-MBP, respectively, whereas for Fc(long)-MBP, 10.54±0.5% of the injected dose remained after 118 hours.

### Example 3

### Antigen Persistence Affects the Proliferation of Antigen-specific T Cells in Vivo

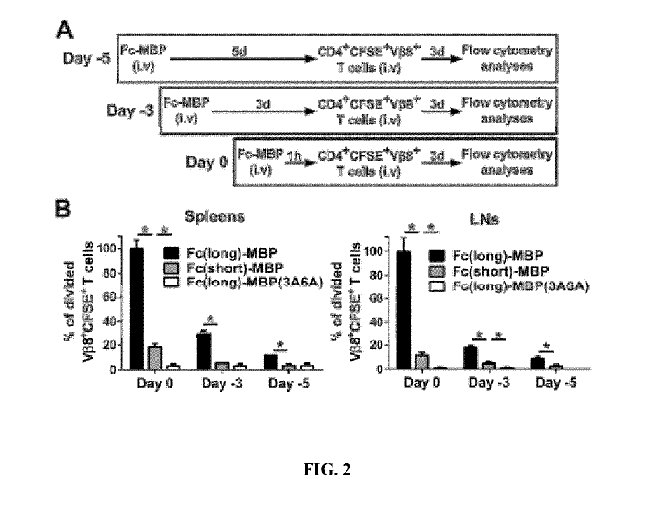

The effect of the distinct properties of the Fc-MBP fusions on the in vivo proliferation of MBP1-91-A^{u}-specific CD4⁺ T cells was next investigated. CFSE-labeled, purified CD4⁺ T cells isolated from MBP1-9:I-A^{u}-specific TCR (Vβ8⁺) transgenic mice were used in adoptive transfers. Prior to T cell transfer into WT B10.PL (I-A^{u}) mice, 1 μg Fc-MBP fusion was injected into recipients on different days (day −5, −3 and 0, referring to 5, 3 and 0 days before the cell transfer, respectively, FIG. 2A). The percentage of divided CD4⁺CFSE⁺Vβ8⁺ T cells was assessed in the spleen and lymph nodes (LNs) three days following T cell transfer. As a control throughout these studies, an Fc-MBP fusion in which the T cell contact residues, Gln3 and Pro6, of the MBP peptide [35] are replaced by Ala [Fc(long)-MBP(3A6A)] was used. Fc(long)-MBP induced higher levels of proliferation in the spleens and LNs than Fc(short)-MBP for all treatments (FIG. 2B). We have previously characterized the properties of Fc(v.short)-MBP in analogous assays [10], and the behavior of Fc(short)-MBP is very similar (FIG. 2B). Fc(long)-MBP(3A6A) induced no detectable proliferative response. Collectively, the data indicate that the increased affinity for FcRn at near neutral pH of Fc(short)-MBP and Fc(v.short)-MBP confers decreased in vivo persistence relative to Fc(long)-MBP, which in turn results in lower T cell responses in vivo (FIG. 2B).

### Example 4

### The Induction of Tolerance Under Prophylactic Conditions is Regulated by Antigen Persistence

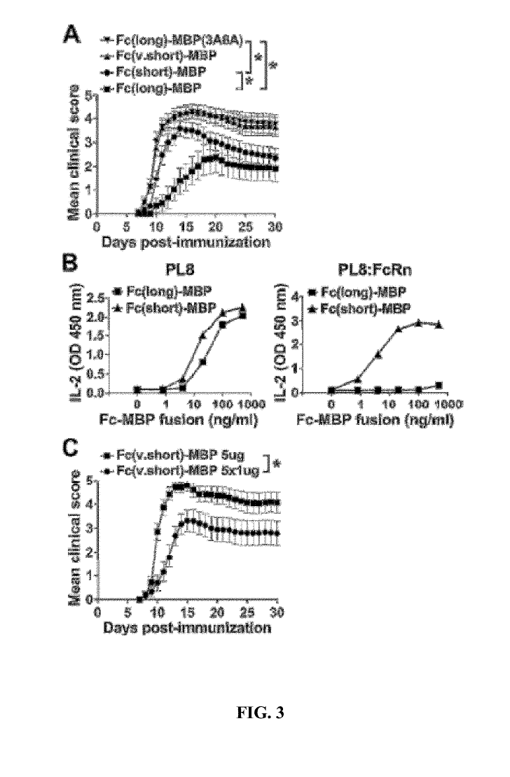

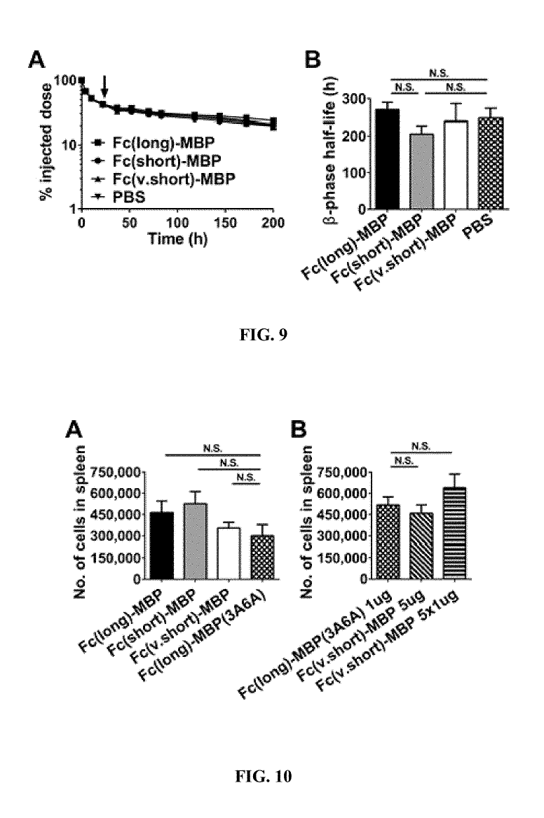

The activity of low doses (1 μg/mouse; ˜50 μg/kg) of the Fc-MBP fusions in inducing T cell tolerance in a prophylactic setting was investigated. These low doses of fusion protein do not affect the activity of FcRn in regulating IgG half-life (FIG. 9). B10.PL mice were pretreated with 1 μg Fc-MBP fusion and immunized 7 days later to induce EAE. Fc(long)-MBP(3A6A) was used as a control. The majority of mice developed either no, or low grade, disease following pretreatment with Fc(long)-MBP (FIG. 3A). Treatment of mice with Fc(short)-MBP was less effective in ameliorating EAE, whereas Fc(v.short)-MBP treatment had no protective effect (FIG. 3A). Thus, low dose antigen induces prophylactic tolerance, but only if antigen persists above a threshold level.

In addition to the shorter half-life of Fc(v. short)-MBP, the inability of Fc(v.short)-MBP to induce tolerance (FIG. 3A) could be due to differences between this fusion and Fc(long)-MBP in endolysosomal trafficking behavior which influences antigen presentation by FcRn-expressing APCs [10, 36]. Specifically, the binding of engineered Fc fragments to FcRn at near neutral pH results in efficient receptor (FcRn)-mediated uptake and accumulation in the endolysosomal pathway in FcRn-expressing cells, by contrast with WT Fc fragments that enter cells by fluid-phase pinocytic processes [11]. Consequently, using FcRn-transfected B lymphoblastoid (PL8:FcRn) [10] cells as APCs, Fc(short)-MBP induced significantly higher IL-2 production by cognate T cell hybridoma (#46 [26]) cells than Fc(long)-MBP (FIG. 3B), whereas in the presence of PL8 cells (that do not express FcRn), the Fc-MBP fusions induced similar levels of cytokine production (FIG. 3B; [10]). Analogously, in earlier studies it was observed that Fc(v.short)-MBP stimulates T cells at around 600-3,000 fold lower concentrations than Fc(long)-MBP in the presence of PL8:FcRn cells [10]. To investigate whether this behavior contributed to the inability of a single dose of Fc(v.short)-MBP to induce tolerance (FIG. 3A), the tolerogenic activity of five doses of 1 μg Fc(v.short)-MBP at 36 hour intervals, starting at 7 days prior to immunization, was compared with a single, equivalent bolus dose (5 μg) delivered at 7 days prior to EAE induction. Importantly, treatment with multiple doses of Fc(v.short)-MBP offered partial protection against EAE, whereas bolus administration of a five-fold higher dose of this Fc-MBP fusion did not affect disease activity (FIG. 3C). These observations indicate that antigen longevity, rather than endolysosomal trafficking behavior, is a dominant factor governing T cell tolerance. In addition, given the relatively small difference in the pharmacokinetic behavior of Fc(short)-MBP and Fc(v.short)-MBP in mice (FIG. 1C), the threshold of antigen persistence necessary for effective prophylaxis is stringent.

### Example 5

### Antigen Specific T Cell Numbers are Reduced During Prophylactic T Cell Tolerance

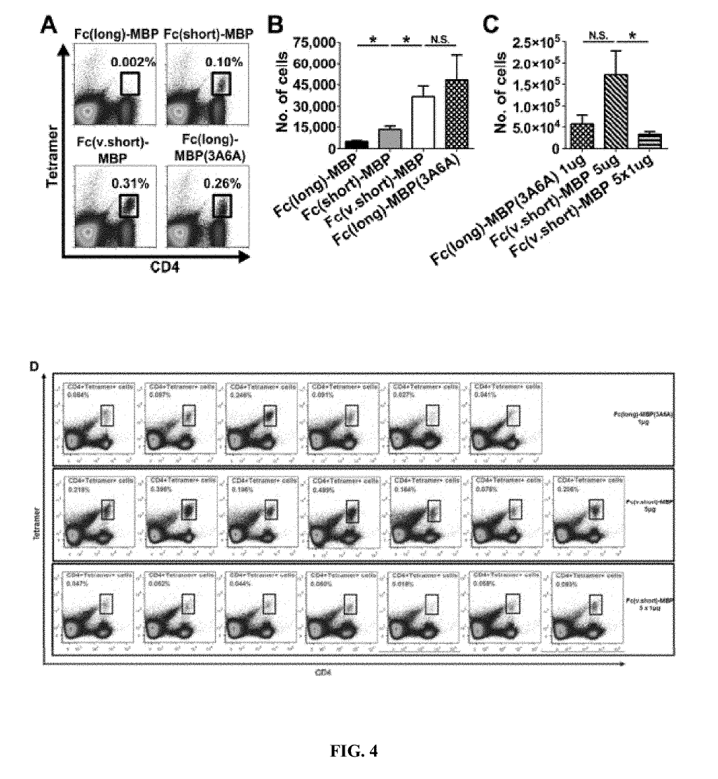

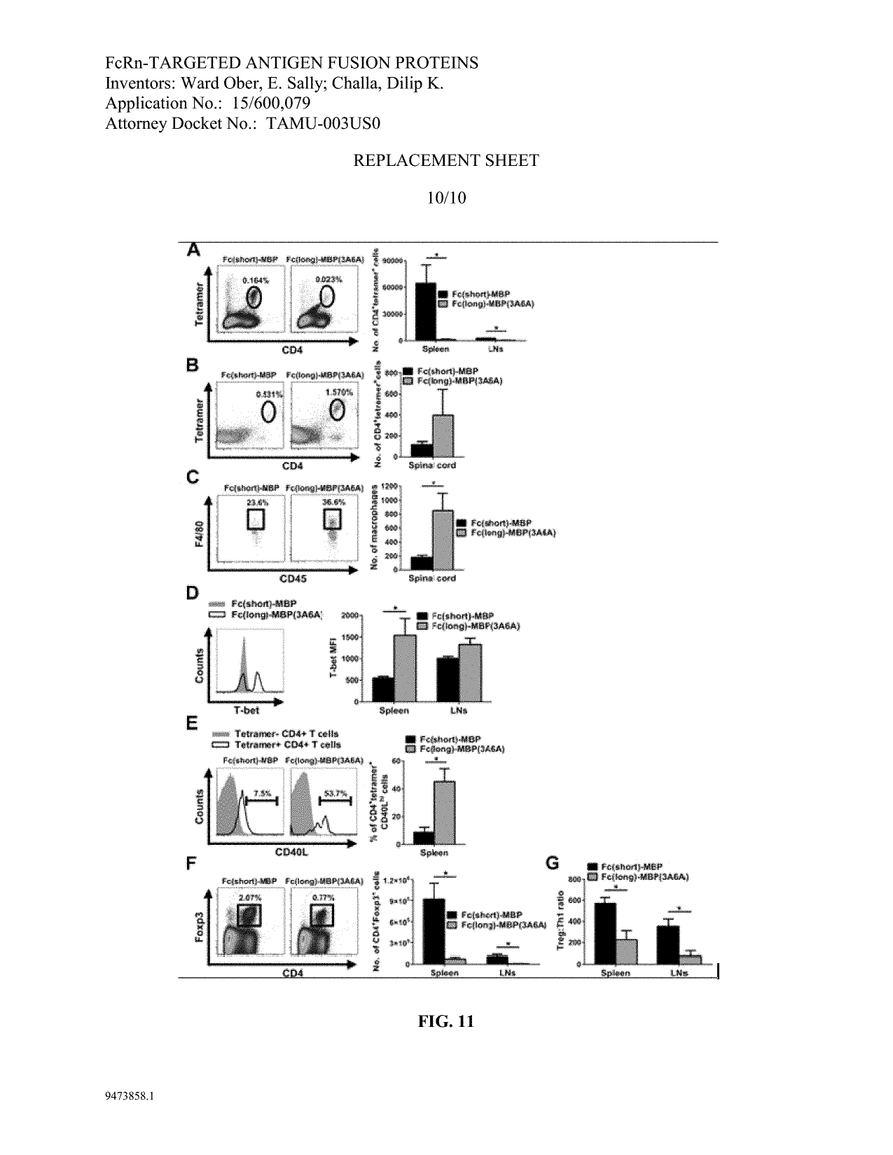

To investigate the mechanism of prophylactic tolerance induction, Fc-MBP fusions were delivered prophylactically and splenic antigen-specific T cells quantitated using fluorescently labeled MBP1-9(4Y)-I-A^{u} tetramers [25] ten days following immunization with MBP1-9. Antigen-specific T cell numbers in the treated mice decreased in the order: Fc(v.short)-MBP (similar to control mice)>Fc(short)-MBP>Fc(long)-MBP (FIGS. 4A, B). In addition, prophylactic delivery of a single dose of 5 μg Fc(v.short)-MBP resulted in higher numbers of antigen-specific T cells compared with treatment using five repeated doses (1 μg/dose) of this Fc-MBP fusion (FIG. 4C). Further, there were no significant differences between the numbers of CD4⁺Foxp3⁺ Tregs in mice treated with the different Fc-MBP fusions (FIG. 10). Consequently, there is a correlation between antigen longevity, disease blockade and reduction in antigen-specific T cell numbers.

### Example 6

### Antigen Persistence Regulates T Cell Tolerance Induction During Ongoing Disease

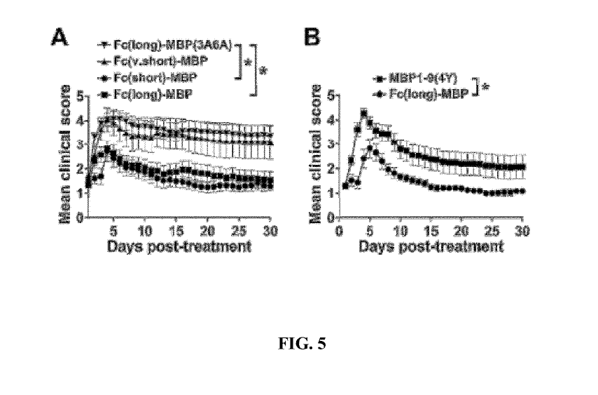

To assess therapeutic tolerance induction, mice were immunized with MBP1-9 to induce EAE and treated with the different fusion proteins (1 μg/mouse; ˜50 μg/kg) following the onset of disease (EAE score of 1-2). Severe disease was observed in the control group of mice within 4-5 days of disease onset, whereas treatment with Fc(long)-MBP resulted in either almost complete recovery or lowered disease to a score of 1-2 following a transient increase in disease score (FIG. 5A). The therapeutic effect of Fc(short)-MBP was analogous to that of Fc(long)-MBP, whereas by analogy with prophylactic tolerance, the treatment of mice with Fc(v.short)-MBP had no effect on ongoing disease. This indicates a requirement for the Fc-MBP fusion to reach a threshold level of persistence for therapeutic tolerance, with the threshold being tightly bounded by the in vivo dynamics of Fc(short)-MBP and Fc(v.short)-MBP (FIG. 1C). Importantly, the delivery of a molar equivalent of MBP1-9(4Y) peptide (33 ng/mouse), which is expected to be rapidly cleared (˜2-30 minutes [5]) by renal filtration, was less effective in treating EAE than Fc(long)-MBP (FIG. 5B).

### Example 7

### The Mechanisms of Prophylactic and Therapeutic Tolerance Induction are Distinct

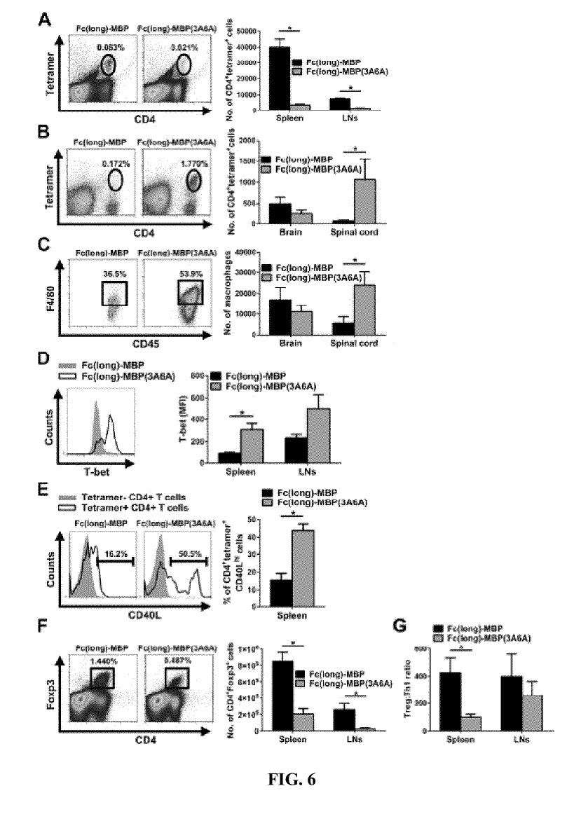

To elucidate the mechanism through which Fc-MBP fusions induce therapeutic tolerance, cells from spleens and draining LNs were analyzed in mice from Fc(long)-MBP and control treatment groups six days following treatment. Unexpectedly, and by marked contrast with the prophylactic setting, the numbers of antigen-specific CD4⁺ T cells in the spleens and LNs of tolerized mice were approximately 10- and 4-fold higher, respectively, than in control mice (FIG. 6A). By contrast, quantitation of the antigen-specific T cells in the brain and spinal cord revealed around 10-fold lower numbers in the spinal cord of Fc(long)-MBP-treated mice, whereas similar numbers were detected in the brain (FIG. 6B). In the majority of murine EAE models, inflammation predominates in the spinal cord rather than the brain [37]. Also, MBP1-9-induced EAE in B10.PL mice is primarily Th1 cell-mediated [38, 39] and it is well established that Th1 cells promote the accumulation of macrophages in the CNS during EAE [40]. Consistent with the reduced T cell infiltrates in the spinal cords of tolerized mice, macrophage numbers were also decreased at this site (FIG. 6C).

The increased numbers of antigen-specific T cells in the periphery of tolerized mice, combined with their reduced numbers in the CNS, prompted us to further characterize these cells by quantitating their levels of the following markers: CXCR3, α4β1, α4β7, LFA-1, CTLA-4, PD-1 and CD40L. In addition, the intracellular levels of the master regulator of Th1 lineage development, T-bet, were analyzed. T-bet and CD40L were the only molecules that were differentially expressed between the groups. T-bet levels were significantly lower in splenic antigen-specific T cells obtained from mice treated with Fc(long)-MBP (FIG. 6D). This trend was also seen in antigen-specific T cells obtained from draining LNs (constituting only ˜20% of the total number of antigen-specific T cells isolated from both spleen and LNs), but the difference was not statistically significant (FIG. 6D). Further, approximately threefold lower numbers of splenic antigen-specific T cells were CD40L^{hi} in Fc(long)-MBP-treated mice by comparison with T cells obtained from control mice (FIG. 6E). Importantly, mice treated with Fc(long)-MBP had higher numbers of CD4⁺Foxp3⁺ Tregs in the spleen and draining LNs (FIG. 6F) which did not bind to MBP1-9(4Y):I-A^{u} tetramers. The increase in CD4⁺Foxp3⁺ Tregs, combined with decrease in CD4⁺T-bet⁺ antigen-specific (Th1) T cells, resulted in higher Treg:Th1 ratios in tolerized mice (FIG. 6G). The treatment of mice with Fc(short)-MBP resulted in similar effects on splenic antigen-specific T cell numbers, their phenotype and CD4⁺Foxp3⁺ Treg numbers (FIG. 11), demonstrating antigen-specific tolerance of splenic T cells combined with the amplification of Tregs in tolerized mice.

### Example 8

The discovery that led to this invention is an unexpected outcome of the above-described experiments aimed at understanding the factors that are important for the induction of T cell tolerance (the opposite of T cell activation). In these experiments, a mouse model of autoimmune disease was employed, in which vaccination (with an emulsion of neuronal antigen and a commonly used adjuvant) is used to activate and proliferate myelin-specific T cells, which attack the host myelin that surrounds the nerves and leads to autoimmune disease. Prophylactic delivery of antigen (same as the one used for vaccination except that no adjuvant is used) prior to vaccination leads to T cell tolerance (opposite of T cell activation) and reduced autoimmune disease symptoms. It was not known how the persistence of antigen (that is delivered prophylactically or prior to vaccination) affects T cell tolerance and disease symptoms. Using antigen delivery vehicles that can be tuned to vary the in vivo persistence of the antigen, embodiments of the invention relate to the finding that an increased in vivo persistence of the prophylactically-administered antigen is a requirement for efficient induction of T cell tolerance (e.g., reduce the efficacy of vaccination and resultant disease symptoms). Embodiments of the invention related to the surprising finding that a reduced in vivo persistence and FcRn-targeting of the prophylactically-administered antigen favors T cell activation (e.g., potentiates the efficacy of vaccines). In contrast to the goal of therapies for autoimmune diseases, the goal of infectious disease and cancer vaccines is to potentiate T cell activation and proliferation. One example of the antigen delivery vehicle was Fc(T252Y/T256E/H433K/N434F)-antigen, where ‘Fc(T252Y/T256E/H433K/N434F)’ is ‘xfcrn’ that both reduces the in vivo persistence of the antigen and targets the antigen in FcRn-dependent fashion. xfcrn-antigen can be used as an adjuvant for infectious disease and cancer vaccines as supported by the observation that administration of Fc(T252Y/T256E/H433K/N434F)-antigen prior to vaccination is superior compared with control fusion proteins in potentiating vaccine-induced antigen-specific T cell proliferation.

The induction of antigen-specific T cell tolerance represents a highly specific approach for the treatment of autoimmunity. However, despite extensive preclinical analyses of the efficacy of immunodominant peptides in tolerance induction, this strategy has met with limited success in the clinic [2, 41-43]. Importantly, the short half-lives of peptides necessitate the use of relatively high doses that can provoke anaphylaxis [6, 7, 44]. Some embodiments of the invention relate to the role of antigen dynamics in tolerance induction, by determining the tolerogenic activity of low doses (˜50 μg/kg) of Fc fusions comprising an immunodominant MBP epitope linked to engineered Fc fragments with different binding properties for FcRn. Other epitopes can be linked to the fragments. These mutated Fc fragments are designed to endow different pharmacokinetic behavior on the appended antigen. In this embodiment, the in vivo persistence of antigen is critical for tolerance induction. The invention also relates to the newly discovered requirement for a stringent threshold of persistence to achieve tolerance in both prophylactic and therapeutic settings.

The in vivo persistence of Fc-MBP fusions is governed by their interactions with FcRn in endothelial cells and/or hematopoietic cells [28]. Amongst hematopoietic cells, all professional APCs express FcRn [10, 14, 36, 45]. Variations in interactions between Fc-MBP fusions and FcRn therefore also regulate epitope loading onto MHC class II molecules and cognate T cell activation. Fc-MBP fusions that are recycled efficiently out of FcRn-expressing cells can lead to poor antigen presentation in vitro, whereas fusions such as Fc(short)-MBP or Fc(v.short)-MBP that bind to FcRn with high affinity at near neutral and acidic pH accumulate to relatively high levels in APCs and are efficiently presented. However, recycled Fc-MBP fusions can have prolonged in vivo persistence, whereas those that accumulate in FcRn-expressing cells can have comparatively short half-lives. Importantly, the induction of tolerance by five doses of Fc(v.short)-MBP delivered over a seven day period prior to EAE induction, combined with the lack of efficacy of an equivalent bolus dose of this fusion protein, demonstrate that the endolysosomal trafficking properties of this protein do not mitigate tolerance induction if antigen persistence is prolonged. In addition, the lack of protection by a single dose of this Fc-MBP fusion indicates a minimum threshold of persistence of low dose antigen for tolerance induction that is tightly bounded by the pharmacokinetic behavior of Fc(short)-MBP and Fc(v.short)-MBP.

There are clinical situations where prophylactic T cell tolerance has potential applications such as the prevention of transplant rejection and reduction of immune responses against protein-based therapeutics [46-48]. In addition, epitope spreading has been observed in patients and animal models of MS [49, 50] and T cells specific for spread epitopes can induce EAE relapses [51]. Consequently, prophylactic tolerization of naive autoreactive T cells specific to potential ‘spreading’ epitopes combined with tolerization of activated autoreactive T cells can result in effective treatment.

By analogy with prophylactic tolerance induction, a threshold of antigen persistence that is delimited by the behavior of Fc(short)-MBP and Fc(v.short)-MBP is also a requirement for the amelioration of ongoing disease. Analyses of the effects of Fc-MBP fusions reveal that although a fusion protein with a shorter persistence (Fc(short)-MBP) is less effective as a tolerogen in the prophylactic setting than its longer lived counterpart, Fc(long)-MBP, both fusion proteins have similar therapeutic activity during EAE. This is possibly due to the different sensitivities of naïve and primed T cells to antigenic stimulation [52]. In addition, the mechanisms of prophylactic and therapeutic tolerance are distinct: prophylactic tolerance induction results in reduced numbers of antigen-specific CD4⁺ T cells in the periphery, indicating T cell deletion or anergy. By contrast, in a therapeutic setting tolerance is unexpectedly accompanied by increased numbers of peripheral antigen-specific CD4⁺ T cells. This contrasts with the induction of T cell apoptosis in mice following the delivery of multiple high doses (400 μg/mouse) of acetylated MBP1-11 following the adoptive transfer of autoreactive CD4⁺ T cells [3].

The increased numbers of splenic antigen-specific T cells in the tolerized mice harbor significantly reduced levels of T-bet, which is essential for the encephalitogenicity of Th1 cells [53] and has been reported to be downregulated in tolerized Th1 cells [54]. In addition, CD40L levels are substantially lower in the majority of splenic antigen-specific T cells in the tolerized mice. Studies using both CD40L knock out mice and anti-CD40L blocking antibodies support a critical role for this molecule in T cell activation and EAE induction or progression [55, 56]. Importantly, the downregulation of T-bet can be a downstream effect of reduced CD40L levels, since CD40L is required for the induction of co-stimulatory molecules such as B7.1 and B7.2 on APCs [55]. Since tolerance induction during active EAE is accompanied by amplification of CD4⁺Foxp3⁺ Tregs, and durable tolerance is dependent on the expansion of Tregs [8, 57], Fc-epitope fusions can have long term effects.

In summary, by using Fc engineering to tune antigen dynamics, some embodiments of the invention relate to a stringent threshold of antigen persistence as a prerequisite for antigen-specific T cell tolerance induction. Low doses of relatively long-lived, Fc-epitope fusions can be effective in ameliorating EAE and similar diseases in both prophylactic and therapeutic settings.

### Example 9

### Recombinant Fc Fragments Fused to Chicken Egg White Ovalbumin (Ova) Peptide—Materials and Methods for Examples 10-11

### Production of Recombinant Proteins

Expression plasmids encoding WT or mutated [T252Y/T256E/H433K/N434F (TT-HN) or H435A] mouse IgG1-derived Fc-hinge connected at the C-termini through a GSGG linker to codons encoding the mutated (K4Y) MBP epitope encompassing residues 1-9 have been described previously [10]. These plasmids were used as templates to generate gene sequences encoding WT or mutated (TT-HN or H435A) mouse IgG1-derived Fc-hinge connected at the C-termini through a GSGG linker to codons encoding the OVA epitope encompassing residues 323-339 using splicing by overlap extension and designed oligonucleotide primers. All Fc-OVA(323-339) fusion genes were cloned into pcDNA™3.4-TOPO® vector (Life Technologies) for expression. The fusion proteins were expressed by transiently transfecting Expi293TM cells (Life Technologies) with the above described Fc-OVA(323-339) fusion protein expression constructs using the Expi293™ expression system kit (Life Technologies). The Fc-OVA fusions were purified from culture supernatants using Ni²⁺-NTA-agarose columns, followed by separation of homodimeric, non-aggregated fractions using size exclusion chromatography (GE Healthcare).

### Surface Plasmon Resonance Analyses

Equilibrium dissociation constants (K_{D}s) for the binding of Fc-OVA(323-339) (WT, TT-HN and H435A) to recombinant mouse FcRn were determined using surface plasmon resonance (BlAcore T200; GE Healthcare) and previously described methods [69]. CM5 chips were coupled with recombinant Fc(WT)-OVA(323-339), Fc(TT-HN)-OVA(323-339), and Fc(H435A)-OVA(323-339) to densities of ˜500-900 RU followed by injection of mFcRn at various concentrations (1-2000 nM) at a flow rate of 10 μl/min using PBS (pH 6.0 or 7.4; Lonza), 0.01% (v/v) Tween-20 as running buffer. Flow cells were regenerated at the end of each cycle using 0.15 M NaCl, 0.1 M sodium bicarbonate (pH 8.5) buffer. FcRn binds to two sites on IgG-Fc that are not equivalent [27]. This results in K_{D} estimates for two dissociation constants, and the values for the higher affinity interaction sites are presented. The data were processed as described previously [27].

### Treatment and Immunization of Mice and Flow Cytometry Analyses

9-10 week old male C57BL/6J mice (The Jackson Laboratory) were injected i.v. with 25 μg Fc-OVA(323-339) fusions (2 mice/group). Seven days later, the mice were immunized subcutaneously at four sites in the flanks with 100 μg Endofit ovalbumin (Invivogen) emulsified with complete Freund's adjuvant (Sigma Aldrich) containing an additional 4 mg/ml heat-inactivated Mycobacterium tuberculosis (strain H37Ra, Becton-Dickinson). Eighteen days post-immunization the mice were euthanized and spleens isolated. Single cell suspensions from spleens were obtained by mechanical disruption and forcing through 70 μm cell strainers (Becton-Dickinson), and cell suspensions were depleted of erythrocytes using red blood cell lysis buffer. To detect OVA(329-337)-specific CD4+ T cells, single cell suspensions (1.25 million) from spleens were incubated with ˜150 μg/ml APC-labeled OVA(329-337)-I-A(b) tetramer or APC-labeled human CLIP(87-101)-I-A(b) control tetramer (both tetramers were obtained from the National Institutes of Health tetramer core facility) in phenol red-free RPMI 1640 medium (Gibco) supplemented with 10% fetal bovine serum (FBS; Gemini Bio-Products), 4 mM GlutaMAXTM (Gibco), 100 U/mL penicillin and 100 μg/mL streptomycin (Gibco) for 135 min at 37° C. Following one wash with cold PBS, the cells were stained with cell viability dye (Zombie Violet, BioLegend) for 30 minutes on ice at a dilution of 1:500 in PBS. Following one wash with cold 5% FBS/PBS, TruStain fcX (BioLegend) was added to the samples at a concentration of 10 μg/ml and incubated on ice for 15 minutes to block Fc receptors. Without washing, the samples were stained with B220-Alexa488 (clone—RA3-6B2; BioLegend), CD4-PE (clone—GK1.5; Becton-Dickinson) and CD44-PerCP-Cy5.5 (clone—IM7; BioLegend) antibodies for 30 minutes on ice. The cells were then washed with cold PBS and fixed using 4% formaldehyde/PBS (Macron Fine Chemicals). The fixed cell samples were analyzed by flow cytometry using LSRFortessa (Becton-Dickinson) and the resulting data was analyzed using FlowJo (FlowJo LLC).

### Example 10

### Binding Properties of Fc-Ova Fusion Proteins

The long in vivo half-life of IgG, Fc or Fc-fusion proteins is governed by the pH-dependent binding between Fc and FcRn [11]. Therefore, two sets of mutations in the Fc region that alter the binding towards FcRn were selected—TT-HN (T252Y/T256E/H433K/N434F) and H435A. TT-HN mutations result in greatly increased binding affinity for FcRn at both pH 6.0 and 7.4 [10, 70], which imparts FcRn-targeting ability and substantially reduced in vivo half-life [70, 28]. The H435A mutation, which ablates the binding of Fc towards FcRn at both physiological and acidic pH, also results in reduced in vivo half-life [28] but Fc or IgG harboring this mutation has no FcRn-targeting ability. Thus, wild-type (WT) or mutant Fc-OVA(323-339) fusion proteins harboring TT-HN or H435A mutation were generated and equilibrium dissociation constants (K_{D}s) for their binding towards mouse FcRn were determined using surface plasmon resonance (Table 2). The KD values indicate that Fc(WT)-OVA(323-339) have long in vivo half-life, whereas both Fc(TT-HN)-OVA(323-339) and Fc(H435A)-OVA(323-339) have short in vivo half-lives with or without FcRn-targeting ability, respectively.

TABLE 2
Binding properties of Fc-OVA(323-339) fusion proteins

Binding to mouse
FcRn (KD, nM)

| Fusion protein | pH 6.0 | pH 7.4 |
|---|---|---|
| Fc(WT)-OVA(323-339) | 299.8 | N.B.* |
| Fc(TT-HN)-OVA(323-339) | 3.0 | 14.9 |
| Fc(H435A)-OVA(323-339) | N.B.* | N.B.* |

*N.B. = no detectable binding.

### Example 11

### Adjuvant Activity of Fc-Ova Fusion Proteins

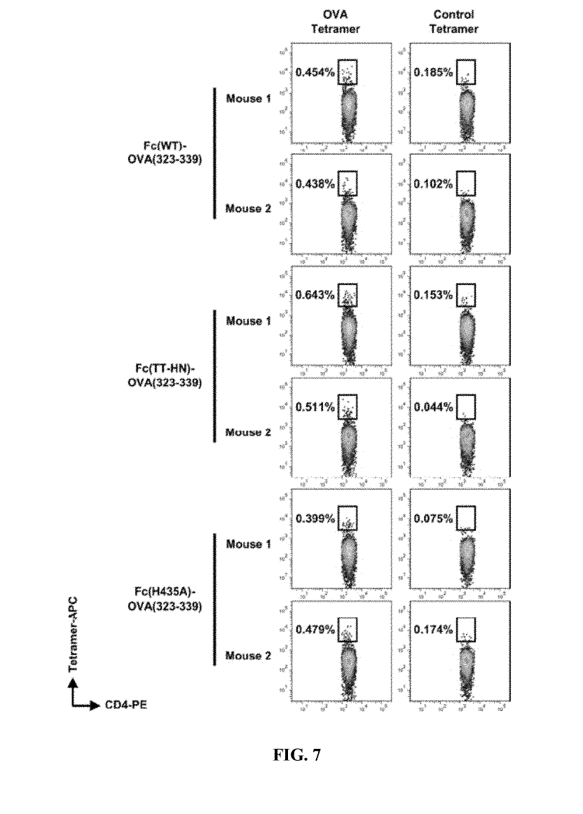

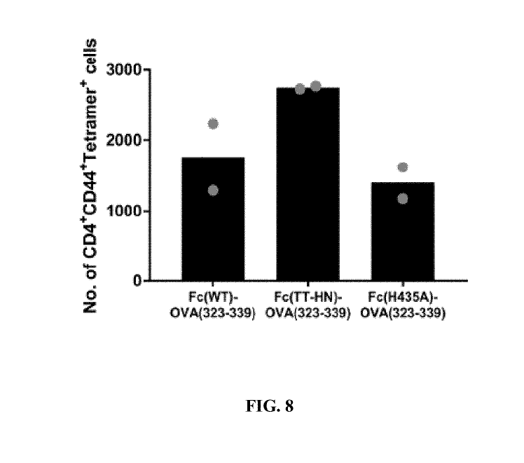

To test the adjuvant activity of the generated fusion proteins, mice were pretreated with relatively low doses (25 μg) of different Fc-OVA(323-339) fusion proteins and seven days later immunized with OVA emulsified in complete Freund's adjuvant. Eighteen days post-immunization, the percentage and total numbers of OVA(329-337)-specific CD4+ T cells in the spleens of different mice were determined using OVA(329-337)-I-A(b) tetramer. As shown in FIG. 7, the percentage of OVA(329-337)-I-A(b) tetramer+cells among CD4+CD44+ T cells was higher in both the mice treated with Fc(TT-HN)-OVA(323-339) in comparison to the mice treated with Fc(WT)-OVA(323-339) or Fc(H435A)-OVA(323-339). In contrast, the staining observed with the control tetramer was at close to background levels and similar between the groups. Further, the total number of splenic CD4+CD44+OVA(329-337)-I-A(b) tetramer+ T cells was greater in mice pretreated with Fc(TT-HN)-OVA(323-339) than that observed in mice treated with the other two fusion proteins (FIG. 8).

In conclusion, the data presented here indicate that prophylactic treatment with FcRn-targeted Fc-antigen (OVA peptide) fusion protein potentiates vaccination-induced antigen (OVA)-specific CD4+ T cell response.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

[1] S. M. Steward-Tharp, Y. J. Song, R. M. Siegel, J. J. O'Shea, New insights into T cell biology and T cell-directed therapy for autoimmunity, inflammation, and immunosuppression, Ann. N. Y. Acad. Sci. 1183 (2010) 123-148.
[2] S. D. Miller, D. M. Turley, J. R. Podojil, Antigen-specific tolerance strategies for the prevention and treatment of autoimmune disease, Nat. Rev. Immunol. 7 (2007) 665-677.
[3] J. M. Critchfield, M. K. Racke, J. C. Zuniga-Pflucker, B. Cannella, C. S. Raine, J. Goverman et al., T cell deletion in high antigen dose therapy of autoimmune encephalomyelitis, Science 263 (1994) 1139-1143.
[4] A. Lutterotti, R. Martin, Antigen-specific tolerization approaches in multiple sclerosis, Expert Opin. Investig. Drugs 23 (2014) 9-20.
[5] S. C. Penchala, M. R. Miller, A. Pal, J. Dong, N. R. Madadi, J. Xie et al., A biomimetic approach for enhancing the in vivo half-life of peptides, Nat. Chem. Biol. 11 (2015) 793-798.
[6] C. E. Smith, T. N. Eagar, J. L. Strominger, S. D. Miller, Differential induction of IgE-mediated anaphylaxis after soluble vs. cell-bound tolerogenic peptide therapy of autoimmune encephalomyelitis, Proc. Natl. Acad. Sci. USA 102 (2005) 9595-9600.
[7] R. Pedotti, D. Mitchell, J. Wedemeyer, M. Karpuj, D. Chabas, E. M. Hattab et al., An unexpected version of horror autotoxicus: anaphylactic shock to a self-peptide, Nat. Immunol. 2 (2001) 216-222.
[8] M. D. Rosenblum, I. K. Gratz, J. S. Paw, K. Lee, A. Marshak-Rothstein, A. K. Abbas, Response to self antigen imprints regulatory memory in tissues, Nature 480 (2011) 538-542.
[9] E. J. Wherry, T cell exhaustion, Nat. Immunol. 12 (2011) 492-499.
[10] W. Mi, S. Wanjie, S. T. Lo, Z. Gan, B. Pickl-Herk, R. J. Ober et al., Targeting the neonatal Fc receptor for antigen delivery using engineered Fc fragments, J. Immunol. 181 (2008) 7550-7561.
[11] E. S. Ward, R. J. Ober, Multitasking by exploitation of intracellular transport functions: the many faces of FcRn, Adv. Immunol. 103 (2009) 77-115.
[12] R. J. Ober, C. Martinez, C. Vaccaro, J. Zhou, E. S. Ward, Visualizing the site and dynamics of IgG salvage by the MHC class I-related receptor, FcRn, J. Immunol. 172 (2004) 2021-2029.
[13] S. M. Claypool, B. L. Dickinson, M. Yoshida, W. I. Lencer, R. S. Blumberg, Functional reconstitution of human FcRn in Madin-Darby canine kidney cells requires co-expressed human β2-microglobulin, J. Biol. Chem. 277 (2002) 28038-28050.
[14] K. Baker, T. Rath, M. Pyzik, R. S. Blumberg, The Role of FcRn in Antigen Presentation, Front. Immunol. 5 (2014) 408.
[15] M. F. Samson, D. E. Smilek, Reversal of acute experimental autoimmune encephalomyelitis and prevention of relapses by treatment with a myelin basic protein peptide analogue modified to form long-lived peptide-MHC complexes, J. Immunol. 155 (1995) 2737-2746.
[16] Z. Jiang, H. Li, D. C. Fitzgerald, G. X. Zhang, A. Rostami, MOG(35-55) i.v suppresses experimental autoimmune encephalomyelitis partially through modulation of Th17 and JAK/STAT pathways, Eur. J. Immunol. 39 (2009) 789-799.
[17] D. R. Getts, A. J. Martin, D. P. McCarthy, R. L. Terry, Z. N. Hunter, W.T. Yap et al., Microparticles bearing encephalitogenic peptides induce T-cell tolerance and ameliorate experimental autoimmune encephalomyelitis, Nat. Biotechnol. 30 (2012) 1217-1224.
[18] C. I. Pearson, H. O. McDevitt, Induction of apoptosis and T helper 2 (Th2) responses correlates with peptide affinity for the major histocompatibility complex in self-reactive T cell receptor transgenic mice, J. Exp. Med. 185 (1997) 583-599.
[19] J. J. Lafaille, K. Nagashima, M. Katsuki, S. Tonegawa, High incidence of spontaneous autoimmune encephalomyelitis in immunodeficient anti-myelin basic protein T cell receptor transgenic mice, Cell 78 (1994) 399-408.
[20] D. Feng, C. J. Bond, L. K. Ely, J. Maynard, K. C. Garcia, Structural evidence for a germline-encoded T cell receptor-major histocompatibility complex interaction ‘codon’, Nat. Immunol. 8 (2007) 975-983.
[21] A. G. Amit, R. A. Mariuzza, S. E. Phillips, R. J. Poljak, Three-dimensional structure of an antigen-antibody complex at 2.8 Å resolution, Science 233 (1986) 747-753.
[22] P. Bansal, T. Khan, U. Bussmeyer, D. K. Challa, R. Swiercz, R. Velmurugan et al., The encephalitogenic, human myelin oligodendrocyte glycoprotein-induced antibody repertoire is directed toward multiple epitopes in C57BL/6-immunized mice, J. Immunol. 191 (2013) 1091-1101.
[23] J. Foote, G. Winter, Antibody framework residues affecting the conformation of the hypervariable loops, J. Mol. Biol. 224 (1992) 487-499.
[24] X. He, C. Radu, J. Sidney, A. Sette, E. S. Ward, K. C. Garcia, Structural snapshot of aberrant antigen presentation linked to autoimmunity: the immunodominant epitope of MBP complexed with I-A^{u}, Immunity 17 (2002) 83-94.
[25] C. G. Radu, S. M. Anderton, M. Firan, D. C. Wraith, E. S. Ward, Detection of autoreactive T cells in H-2^{u} mice using peptide-MHC multimers, Int. Immunol. 12 (2000) 1553-1560.
[26] J. C. Huang, M. Han, A. Minguela, S. Pastor, A. Qadri, E. S. Ward, T cell recognition of distinct peptide:I-A^{u} conformers in murine experimental autoimmune encephalomyelitis, J. Immunol. 171 (2003) 2467-2477.
[27] J. Zhou, F. Mateos, R. J. Ober, E. S. Ward, Conferring the binding properties of the mouse MHC class I-related receptor, FcRn, onto the human ortholog by sequential rounds of site-directed mutagenesis, J. Mol. Biol. 345 (2005) 1071-1081.
[28] H. Perez-Montoyo, C. Vaccaro, M. Hafner, R. J. Ober, W. Mueller, E. S. Ward, Conditional deletion of the MHC Class I-related receptor, FcRn, reveals the sites of IgG homeostasis in mice, Proc. Natl. Acad. Sci. USA 106 (2009) 2788-2793.
[29] C. Vaccaro, J. Zhou, R. J. Ober, E. S. Ward, Engineering the Fc region of immunoglobulin G to modulate in vivo antibody levels, Nat. Biotechnol. 23 (2005) 1283-1288.
[30] V. Ghetie, S. Popov, J. Borvak, C. Radu, D. Matesoi, C. Medesan et al., Increasing the serum persistence of an IgG fragment by random mutagenesis, Nat. Biotechnol. 15 (1997) 637-640.
[31] R. L. Shields, A. K. Namenuk, K. Hong, Y. G. Meng, J. Rae, J. Briggs et al., High resolution mapping of the binding site on human IgG1 for FcγRI, FcγRII, FcγRIII, and FcRn and design of IgG1 variants with improved binding to the FcγR, J. Biol. Chem. 276 (2001) 6591-6604.
[32] C. Vaccaro, R. Bawdon, S. Wanjie, R. J. Ober, E. S. Ward, Divergent activities of an engineered antibody in murine and human systems have implications for therapeutic antibodies, Proc. Natl. Acad. Sci. USA 103 (2006) 18709-18714.
[33] G. Y. Liu, P. J. Fairchild, R. M. Smith, J. R. Prowle, D. Kioussis, D.C. Wraith, Low avidity recognition of self-antigen by T cells permits escape from central tolerance, Immunity 3 (1995) 407-415.
[34] A. Seamons, J. Sutton, D. Bai, E. Baird, N. Bonn, B. F. Kafsack et al., Competition between two MHC binding registers in a single peptide processed from myelin basic protein influences tolerance and susceptibility to autoimmunity, J. Exp. Med. 197 (2003) 1391-1397.
[35] D. C. Wraith, B. Bruun, P. J. Fairchild, Cross-reactive antigen recognition by an encephalitogenic T cell receptor. Implications for T cell biology and autoimmunity, J. Immunol. 149 (1992) 3765-3770.
[36] S. W. Qiao, K. Kobayashi, F. E. Johansen, L. M. Sollid, J. T. Andersen, E. Milford et al., Dependence of antibody-mediated presentation of antigen on FcRn, Proc. Natl. Acad. Sci. USA 105 (2008) 9337-9342.
[37] A. K. Wensky, G. C. Furtado, M. C. Marcondes, S. Chen, D. Manfra, S.A. Lira et al., IFN-γ determines distinct clinical outcomes in autoimmune encephalomyelitis, J. Immunol. 174 (2005) 1416-1423.
[38] L. Gabrysova, D. C. Wraith, Antigenic strength controls the generation of antigen-specific IL-10-secreting T regulatory cells, Eur. J. Immunol. 40 (2010) 1386-1395.
[39] D. G. Ando, J. Clayton, D. Kono, J. L. Urban, E. E. Sercarz, Encephalitogenic T cells in the B10.PL model of experimental allergic encephalomyelitis (EAE) are of the Th-1 lymphokine subtype, Cell. Immunol. 124 (1989) 132-143.
[40] D. R. Huang, J. Wang, P. Kivisakk, B. J. Rollins, R. M. Ransohoff, Absence of monocyte chemoattractant protein 1 in mice leads to decreased local macrophage recruitment and antigen-specific T helper cell type 1 immune response in experimental autoimmune encephalomyelitis, J. Exp. Med. 193 (2001) 713-726.
[41] K. G. Warren, I. Catz, L. Z. Ferenczi, M. J. Krantz, Intravenous synthetic peptide MBP8298 delayed disease progression in an HLA Class II-defined cohort of patients with progressive multiple sclerosis: results of a 24-month double-blind placebo-controlled clinical trial and 5 years of follow-up treatment, Eur. J. Neurol. 13 (2006) 887-895.
[42] M. S. Freedman, A. Bar-Or, J. Oger, A. Traboulsee, D. Patry, C. Young et al., A phase III study evaluating the efficacy and safety of MBP8298 in secondary progressive MS, Neurology 77 (2011) 1551-1560.
[43] B. Bielekova, B. Goodwin, N. Richert, I. Cortese, T. Kondo, G. Afshar et al., Encephalitogenic potential of the myelin basic protein peptide (amino acids 83-99) in multiple sclerosis: results of a phase II clinical trial with an altered peptide ligand, Nat. Med. 6 (2000) 1167-1175.
[44] E. Liu, H. Moriyama, N. Abiru, D. Miao, L. Yu, R. M. Taylor et al., Anti-peptide autoantibodies and fatal anaphylaxis in NOD mice in response to insulin self-peptides B:9-23 and B:13-23, J. Clin. Invest. 110 (2002) 1021-1027.
[45] X. Zhu, G. Meng, B. L. Dickinson, X. Li, E. Mizoguchi, L. Miao et al., MHC class I-related neonatal Fc receptor for IgG is functionally expressed in monocytes, intestinal macrophages, and dendritic cells, J. Immunol. 166 (2001) 3266-3276.
[46] A. S. De Groot, D. W. Scott, Immunogenicity of protein therapeutics, Trends Immunol. 28 (2007) 482-490.
[47] J. A. Bluestone, H. Auchincloss, G. T. Nepom, D. Rotrosen, E. W. St Clair, L. A. Turka, The Immune Tolerance Network at 10 years: tolerance research at the bedside, Nat. Rev. Immunol. 10 (2010) 797-803.
[48] S. Krishnamoorthy, T. Liu, D. Drager, S. Patarroyo-White, E. S. Chhabra, R. Peters et al., Recombinant factor VIII Fc (rFVIIIFc) fusion protein reduces immunogenicity and induces tolerance in hemophilia A mice, Cell. Immunol. 301 (2016) 30-39.
[49] V. K. Tuohy, M. Yu, L. Yin, J. A. Kawczak, R. P. Kinkel, Spontaneous regression of primary autoreactivity during chronic progression of experimental autoimmune encephalomyelitis and multiple sclerosis, J. Exp. Med. 189 (1999) 1033-1042.
[50] N. Goebels, H. Hofstetter, S. Schmidt, C. Brunner, H. Wekerle, R. Hohlfeld, Repertoire dynamics of autoreactive T cells in multiple sclerosis patients and healthy subjects: epitope spreading versus clonal persistence, Brain 123 (2000) 508-518.
[51] S. L. Bailey, B. Schreiner, E. J. McMahon, S. D. Miller, CNS myeloid DCs presenting endogenous myelin peptides ‘preferentially’ polarize CD4⁺ T(H)-17 cells in relapsing EAE, Nat. Immunol. 8 (2007) 172-180.
[52] G. Iezzi, K. Karjalainen, A. Lanzavecchia, The duration of antigenic stimulation determines the fate of naive and effector T cells, Immunity 8 (1998) 89-95.
[53] Y. Yang, J. Weiner, Y. Liu, A. J. Smith, D. J. Huss, R. Winger et al., T-bet is essential for encephalitogenicity of both Th1 and Th17 cells, J. Exp. Med. 206 (2009) 1549-1564.
[54] M. Long, A. M. Slaiby, A. T. Hagymasi, M. A. Mihalyo, A. C. Lichtler, S. L. Reiner et al., T-bet down-modulation in tolerized Th1 effector CD4 cells confers a TCR-distal signaling defect that selectively impairs IFN-γ expression, J. Immunol. 176 (2006) 1036-1045.
[55] I. S. Grewal, H. G. Foellmer, K. D. Grewal, J. Xu, F. Hardardottir, J.L. Baron et al., Requirement for CD40 ligand in costimulation induction, T cell activation, and experimental allergic encephalomyelitis, Science 273 (1996) 1864-1867.
[56] K. Gerritse, J. D. Laman, R. J. Noelle, A. Aruffo, J. A. Ledbetter, W. J. Boersma et al., CD4O-CD40 ligand interactions in experimental allergic encephalomyelitis and multiple sclerosis, Proc. Natl. Acad. Sci. USA 93 (1996) 2499-2504.
[57] D. R. Getts, D. M. Turley, C. E. Smith, C. T. Harp, D. McCarthy, E.M. Feeney et al., Tolerance induced by apoptotic antigen-coupled leukocytes is induced by PD-L1⁺ and IL-10-producing splenic macrophages and maintained by T regulatory cells, J. Immunol. 187 (2011) 2405-2417.
[58] M. Venkataraman, M. Aldo-Benson, Y. Borel, D. W. Scott, Persistence of antigen-binding cells with surface tolerogen: isologous versus heterologous immunoglobulin carriers, J. Immunol. 119 (1977) 1006-1009.
[59] L. P. Cousens, R. Tassone, B. D. Mazer, V. Ramachandiran, D. W. Scott, A. S. De Groot, Tregitope update: mechanism of action parallels IVIg, Autoimmun. Rev. 12 (2013) 436-443
[60] M. A. Sekeres, H. Kantarjian, P. Fenaux, P. Becker, A. Boruchov, A. Guerci-Bresler et al., Subcutaneous or intravenous administration of romiplostim in thrombocytopenic patients with lower risk myelodysplastic syndromes, Cancer 117 (2011) 992-1000.
[61] A. Kavanaugh, I. McInnes, P. Mease, G. G. Krueger, D. Gladman, J. Gomez-Reino et al., Golimumab, a new human tumor necrosis factor alpha antibody, administered every four weeks as a subcutaneous injection in psoriatic arthritis: Twenty-four-week efficacy and safety results of a randomized, placebo-controlled study, Arthritis Rheum. 60 (2009) 976-986.
[62] S. B. Hanauer, W. J. Sandborn, P. Rutgeerts, R. N. Fedorak, M. Lukas, D. MacIntosh et al., Human anti-tumor necrosis factor monoclonal antibody (adalimumab) in Crohn's disease: the CLASSIC-I trial, Gastroenterology 130 (2006) 323-333.
[63] K. A. Papp, S. Tyring, M. Lahfa, J. Prinz, C. E. Griffiths, A. M. Nakanishi et al., A global phase III randomized controlled trial of etanercept in psoriasis: safety, efficacy, and effect of dose reduction, Br. J. Dermatol. 152 (2005) 1304-1312.
[64] W. F. Dall'Acqua, P. A. Kiener, H. Wu, Properties of human IgGls engineered for enhanced binding to the neonatal Fc receptor (FcRn), J. Biol. Chem. 281 (2006) 23514-23524.
[65] I. Metz, S. D. Weigand, B. F. Popescu, J. M. Frischer, J. E. Parisi, Y. Guo et al., Pathologic heterogeneity persists in early active multiple sclerosis lesions, Ann. Neurol. 75 (2014) 728-738.
[66] J. M. Frischer, S. Bramow, A. Dal-Bianco, C. F. Lucchinetti, H. Rauschka, M. Schmidbauer et al., The relation between inflammation and neurodegeneration in multiple sclerosis brains, Brain 132 (2009) 1175-1189.
[67] S. S. Zamvil, D. J. Mitchell, A. C. Moore, K. Kitamura, L. Steinman, J.B. Rothbard, T-cell epitope of the autoantigen myelin basic protein that induces encephalomyelitis, Nature 324 (1986) 258-260.
[68] D. Furman, M. M. Davis, New approaches to understanding the immune response to vaccination and infection, Vaccine 33 (2015) 5271-5281.
[69] R. J. Ober & E. S. Ward, Compensation for loss of ligand activity in surface plasmon resonance experiments. Anal. Biochem. 306, 228-236 (2002).
[70] D. K. Challa, W. Mi, S. T. Lo, R. J. Ober & E. S. Ward, Antigen dynamics govern the induction of CD4(+) T cell tolerance during autoimmunity. J. Autoimmun. 72, 84-94 (2016).
[71] P. R. Hinton, J. M. Xiong, M. G. Johlfs, M. T. Tang, S. Keller & N. Tsurushita, An Engineered Human IgG1 Antibody with Longer Serum Half-Life. J. Immunol. 176, 346-356 (2006).

What is claimed is:

1. A ligand-antigen fusion protein which induces or potentiates immune activation, including the expansion of antigen-specific T cells, wherein the ligand is capable of binding to a Fc receptor (FcRn).

2. The ligand-antigen fusion of claim 1 wherein the ligand is selected from the group consisting of Fc, IgG, single chain Fv, and a nanobody or any other protein that binds to FcRn.

3. The ligand-antigen fusion of claim 1 wherein the ligand is engineered so that it binds to the FcRn in a pH-independent manner.

4. The ligand-antigen fusion of claim 1 wherein the ligand is an engineered Fc region.

5. The ligand-antigen fusion of claim 1 wherein the antigen is an immunodominant epitope.

6. The ligand-antigen fusion of claim 1 wherein multiple different antigens are fused to the ligand.

7. The ligand-antigen fusion of claim 1 wherein multiple immunodominant epitopes are fused to the ligand.

8. The ligand-antigen fusion of claim 1 wherein multiple immunodominant epitopes and antigens are fused to the ligand.

9. The ligand-antigen fusion of claim 5 wherein the immunodominant epitope is a myelin basic protein peptide (MBP1-9).

10. An antigen delivery vehicle for vaccines comprising a ligand-antigen fusion protein, wherein the ligand is capable of binding to a Fc receptor (FcRn).

11. A method of treating a subject with cancer or infectious disease comprising administration of the ligand-antigen fusion of claim 1 to the subject.

12. The method of claim 11, wherein the ligand-antigen fusion is administered as one or more doses following administration of the vaccine.

13. The method of claim 11, wherein the ligand-antigen fusion is administered as one or more doses prior to administration of the vaccine.

14. The method of claim 11, wherein the ligand-antigen fusion is administered as one or more doses prior to and following administration of the vaccine.

15. The method of claim 11, wherein administration of the ligand-antigen fusion results in T cell expansion and/or activation.

16. A ligand-antigen fusion containing a pattern recognition receptor ligand that induces or potentiates immune activation.

17. A ligand-antigen fusion containing a cytokine that induces or potentiates immune activation.


SEQUENCE LISTING (14 sequences)

SEQ ID NO: 1 - DNA, 831 bp, Mouse
atgggatggagctgtatcatcctcttcttggtagcaacagctacaggtgtccactccgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaagggatcaggcggtatcagccaggctgttcacgcagctcacgccgagatcaacgaggctggtaggggatcaggccatcaccatcaccatcactaa

SEQ ID NO: 2 - DNA, 831 bp, Mouse
atgggatggagctgtatcatcctcttcttggtagcaacagctacaggtgtccactccgtgcccagggattgtggttgtaagccttgcatatgtacagtcccagaagtatcatctgtcttcatcttccccccaaagcccaaggatgtgctctacattactctggaacctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgaaattccaccatactgagaagagcctctcccactctcctggtaagggatcaggcggtatcagccaggctgttcacgcagctcacgccgagatcaacgaggctggtaggggatcaggccatcaccatcaccatcactaa

SEQ ID NO: 3 - DNA, 831 bp, Mouse
atgggatggagctgtatcatcctcttcttggtagcaacagctacaggtgtccactccgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaacgcacatactgagaagagcctctcccactctcctggtaagggatcaggcggtatcagccaggctgttcacgcagctcacgccgagatcaacgaggctggtaggggatcaggccatcaccatcaccatcactaa

SEQ ID NO: 4 - DNA, 846 bp, Human
atgggatggagctgtatcatcctcttcttggtagcaacagctacaggtgtccactccgagcccaagagctgcgacaagacccacacctgccccccctgccccgcccccgagctgctgggcggccccagcgtgttcctgttcccccccaagcccaaggacaccctgtacatcactcgcgaacccgaggtgacctgcgtggtggtggacgtgagccacgaggaccccgaggtgaagttcaactggtacgtggacggcgtggaggtgcacaacgccaagaccaagccccgcgaggagcagtacaacagcacctaccgcgtggtgagcgtgctgaccgtgctgcaccaggactggctgaacggcaaggagtacaagtgcaaggtgagcaacaaggccctgcccgcccccatcgagaagaccatcagcaaggccaagggccagccccgcgagccccaggtgtacaccctgccccccagccgcgacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccgctggcagcagggcaacgtgttcagctgcagcgtgatgcacgaggccctgaaattccactacacccagaagagcctgagcctgagccccggcaagggatcaggcggtatcagccaggctgttcacgcagctcacgccgagatcaacgaggctggtaggggatcaggccatcaccatcaccatcactaa

SEQ ID NO: 5 - DNA, 834 bp, Human
atgggatggagctgtatcatcctcttcttggtagcaacagctacaggtgtccactccgagcgcaagtgctgcgtggagtgccccccctgccccgccccccccgtggccggccccagcgtgttcctgttcccccccaagcccaaggacaccctgtacatcactcgcgaacccgaggtgacctgcgtggtggtggacgtgagccacgaggaccccgaggtgcagttcaactggtacgtggacggcgtggaggtgcacaacgccaagaccaagccccgcgaggagcagttcaacagcaccttccgcgtggtgagcgtgctgaccgtggtgcaccaggactggctgaacggcaaggagtacaagtgcaaggtgagcaacaagggcctgcccgcccccatcgagaagaccatcagcaagaccaagggccagccccgcgagccccaggtgtacaccctgccccccagccgcgaggagatgaccaagaaccaggtgagcctgacctgcctggtgaagggcttctaccccagcgacatcgccgtggagtgggagagcaacggccagcccgagaacaactacaagaccaccccccccatgctggacagcgacggcagcttcttcctgtacagcaagctgaccgtggacaagagccgctggcagcagggcaacgtgttcagctgcagcgtgatgcacgaggccctgaaattccactacacccagaagagcctgagcctgagccccggcaagggatcaggcggtatcagccaggctgttcacgcagctcacgccgagatcaacgaggctggtaggggatcaggccatcaccatcaccatcactaa

SEQ ID NO: 6 - DNA, 987 bp, Human
atgggatggagctgtatcatcctcttcttggtagcaacagctacaggtgtccactccgagctgaagacccccctgggcgacaccacccacacctgcccccgctgccccgagcccaagagctgcgacaccccccccccctgcccccgctgccccgagcccaagagctgcgacaccccccccccctgcccccgctgccccgagcccaagagctgcgacaccccccccccctgcccccgctgccccgcccccgagctgctgggcggccccagcgtgttcctgttcccccccaagcccaaggacaccctgtacatcactcgcgaacccgaggtgacctgcgtggtggtggacgtgagccacgaggaccccgaggtgcagttcaagtggtacgtggacggcgtggaggtgcacaacgccaagaccaagccccgcgaggagcagtacaacagcaccttccgcgtggtgagcgtgctgaccgtgctgcaccaggactggctgaacggcaaggagtacaagtgcaaggtgagcaacaaggccctgcccgcccccatcgagaagaccatcagcaagaccaagggccagccccgcgagccccaggtgtacaccctgccccccagccgcgaggagatgaccaagaaccaggtgagcctgacctgcctggtgaagggcttctaccccagcgacatcgccgtggagtgggagagcagcggccagcccgagaacaactacaacaccaccccccccatgctggacagcgacggcagcttcttcctgtacagcaagctgaccgtggacaagagccgctggcagcagggcaacatcttcagctgcagcgtgatgcacgaggccctgaaattccgcttcacccagaagagcctgagcctgagccccggcaagggatcaggcggtatcagccaggctgttcacgcagctcacgccgagatcaacgaggctggtaggggatcaggccatcaccatcaccatcactaa

SEQ ID NO: 7 - DNA, 837 bp, Human
atgggatggagctgtatcatcctcttcttggtagcaacagctacaggtgtccactccgagagcaagtacggccccccctgccccagctgccccgcccccgagttcctgggcggccccagcgtgttcctgttcccccccaagcccaaggacaccctgtacatcactcgcgaacccgaggtgacctgcgtggtggtggacgtgagccaggaggaccccgaggtgcagttcaactggtacgtggacggcgtggaggtgcacaacgccaagaccaagccccgcgaggagcagttcaacagcacctaccgcgtggtgagcgtgctgaccgtgctgcaccaggactggctgaacggcaaggagtacaagtgcaaggtgagcaacaagggcctgcccagcagcatcgagaagaccatcagcaaggccaagggccagccccgcgagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccgcctgaccgtggacaagagccgctggcaggagggcaacgtgttcagctgcagcgtgatgcacgaggccctgaaattccactacacccagaagagcctgagcctgagcctgggcaagggatcaggcggtatcagccaggctgttcacgcagctcacgccgagatcaacgaggctggtaggggatcaggccatcaccatcaccatcactaa

SEQ ID NO: 8 - protein, 276 aa, Mouse
MGWSCIILFLVATATGVHSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKGSGGISQAVHAAHAEINEAGRGSGHHHHHH

SEQ ID NO: 9 - protein, 276 aa, Mouse
MGWSCIILFLVATATGVHSVPRDCGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLKFHHTEKSLSHSPGKGSGGISQAVHAAHAEINEAGRGSGHHHHHH

SEQ ID NO: 10 - protein, 276 aa, Mouse
MGWSCIILFLVATATGVHSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNAHTEKSLSHSPGKGSGGISQAVHAAHAEINEAGRGSGHHHHHH

SEQ ID NO: 11 - protein, 281 aa, Human
MGWSCIILFLVATATGVHSEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGKGSGGISQAVHAAHAEINEAGRGSGHHHHHH

SEQ ID NO: 12 - protein, 277 aa, Human
MGWSCIILFLVATATGVHSERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALKFHYTQKSLSLSPGKGSGGISQAVHAAHAEINEAGRGSGHHHHHH

SEQ ID NO: 13 - protein, 328 aa, Human
MGWSCIILFLVATATGVHSELKTPLGDTTHTCPRCPEPKSCDTPPPCPRCPEPKSCDTPPPCPRCPEPKSCDTPPPCPRCPAPELLGGPSVFLFPPKPKDTLYITREPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALKFRFTQKSLSLSPGKGSGGISQAVHAAHAEINEAGRGSGHHHHHH

SEQ ID NO: 14 - protein, 278 aa, Human
MGWSCIILFLVATATGVHSESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALKFHYTQKSLSLSLGKGSGGISQAVHAAHAEINEAGRGSGHHHHHH